(19) United States
(12) Patent Application Publication
(10) Pub. No.: US 2009/0221640 A1
(43) Pub. Date: Sep. 3, 2009

(54) Novel Crystal Modifications

(75) Inventors: Lars-Erik Briggner, Lund (SE); Anders Eriksson, Lund (SE); Neil Barnwell, Loughborough (GB); Andrea Cole, Loughborough (GB); Jacob Perkins, Loughborough (GB); Luis-Manuel Vaz, Loughborough (GB); Andrew Wells, Loughborough (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/282,974

(22) PCT Filed: Mar. 15, 2007
(86) PCT No.: PCT/SE2007/000256

§ 371 Date: Mar. 16, 2009

Related U.S. Application Data
(60) Provisional application No. 60/782,979, filed on Mar. 16, 2006.

(51) Int. Cl. A61K 31/4545; C07D 213/02; C12P 17/10; C07C 229/00; A61P 29/00

(52) U.S. Cl. 514/318; 546/193; 435/121; 560/37

(57) ABSTRACT

Novel crystal modifications of (5S)-5-[4-(5-chloro-pyridin-2-yloxy)-piperidine-1-sulfonylmethyl]-5-methyl-imidazolidine-2,4-dione are disclosed together with processes for preparing such modifications, pharmaceutical compositions comprising such a modification, and the use of such a modification in therapy.

### FIELD OF THE INVENTION

The present invention discloses novel crystal modifications of (5S)-5-[4-(5-chloro-pyridin-2-yloxy)-piperidine-1-sulfonylmethyl]-5-methyl-imidazolidine-2,4-dione, processes for preparing such modifications, pharmaceutical compositions comprising such a modification, and the use of such a modification in therapy.

### BACKGROUND OF THE INVENTION

WO 02/074767, which is incorporated herein by reference in its entirety, teaches a class of metalloproteinase inhibitors that are useful in therapy.

WO 02/074767 further discloses a specific metalloproteinase inhibitor compound identified therein as (5S)-5-[4-(5-chloro-pyridin-2-yloxy)-piperidine-1-sulfonylmethyl]-5-methyl-imidazolidine-2,4-dione (page 65, lines 15 to 27; and page 120, lines 23 to 29). This compound is designated herein as compound (I).

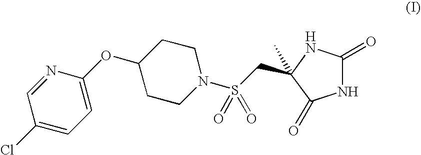

WO 02/074767 further discloses processes for the preparation of compound (I).

Thus, in one embodiment, compound (I) is prepared by a route analogous to that shown in the following Scheme (WO 02/074767; pages 87, 113 and 120) but substituting the appropriate amine in step (d):

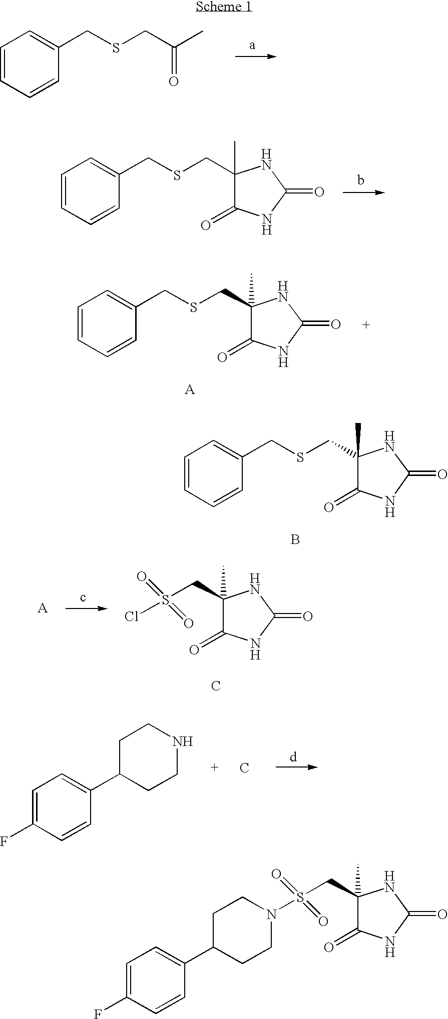

Reagents and conditions for Scheme 1: a) KCN, (NH₄)₂CO₃, EtOH/H₂O, +90° C., 3 h; b) Chiral separation, CHIRALPAK AD, methanol as eluent; c) Cl₂ (g), AcOH/H₂O, <+15° C., 25 min; d) Diisopropylethylamine, THF. −20° C., 30 min.

The obtained compound (I) is then purified either by precipitation and washing with ethanol/water or by preparative HPLC.

In a second embodiment, the racemate of compound (I), (5RS)-5-[4-(5-chloro-pyridin-2-yloxy)-piperidine-1-sulfonylmethyl]-5-methyl-imidazolidine-2,4-dione, was prepared by reacting 1-[4-(5-chloro-pyridin-2-yloxy)-piperidine-1-sulfonyl]-propan-2-one with an excess of potassium cyanide and ammonium carbonate in ethanol, and isolating the product by precipitation. Compound (I), the (5S)-enantiomer, was then obtained by chiral HPLC (WO 02/074767; pages 55 and 65).

No crystalline forms of compound (I) are disclosed in WO 02/074767.

Compound (I) is a potent metalloproteinase inhibitor, particularly a potent inhibitor of MMP12, and as such is useful in therapy. However, when made according to the processes described in WO 02/074767, compound (I) exhibits unpredictable solid state properties with respect to thermodynamic stability. To prepare pharmaceutical formulations containing compound (I) for administration to humans in accordance with the requirements of U.S. and other international health registration authorities, there is a need to produce compound (I) in a stable form, such as a stable crystalline form, having constant physical properties.

Polymorphism can be characterised as the ability of a particular compound to crystallise in different crystal modifications whilst maintaining the same chemical formula. Polymorphs of a given substance are chemically identical in containing the same atoms bonded to one another in the same way, but differ in their crystal modifications, which can affect one or more physical properties such as dissolution rate, melting point, bulk density, stability, flow properties, etc. As used in the specification with reference to a specific compound, the terms “polymorph”, “crystal modification”, “crystal form”, “crystalline modification” and “(crystalline) Form” are to be understood as synonymous.

The present invention provides a method to improve the thermodynamic properties of compound (I) in the solid state, and thereby provides compound (I) in a stable crystalline modification which has consistent and advantageous physical properties.

### BRIEF DESCRIPTION OF THE DRAWINGS

FIG. 1 is an X-ray powder diffraction diagram of compound (I) Form G;

FIG. 2 is a differential scanning calorimetry (DSC) trace and a thermal gravimetric analytical (TGA) trace of compound (I) Form G;

FIG. 3 is an X-ray powder diffraction diagram of compound (I) Form A;

FIG. 4 is an X-ray powder diffraction diagram of compound (I) Form B;

FIG. 5 is an X-ray powder diffraction diagram of compound (I) Form C;

FIG. 6 is an X-ray powder diffraction diagram of compound (I) Form D;

FIG. 7 is an X-ray powder diffraction diagram of compound (I) Form E;

FIG. 8 is an X-ray powder diffraction diagram of compound (I) Form F;

### DISCLOSURE OF THE INVENTION

It has now surprisingly been found that (5S)-5-[4-(5-chloro-pyridin-2-yloxy)-piperidine-1-sulfonylmethyl]-5-methyl-imidazolidine-2,4-dione, compound (I), can exist in at least seven distinct crystalline modifications (polymorphs).

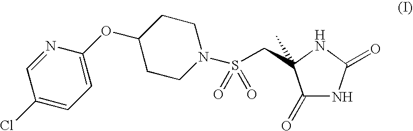

In one aspect, the present invention provides seven polymorphic forms of the compound of formula (I).

In one embodiment, the invention provides a crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern comprising specific peaks at 10.1, 16.2, 16.8 and 19.0 °2θ and wherein said XPRD pattern is measured using CuK_{α} radiation.

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern comprising specific peaks at 9.7, 10.1, 11.5, 12.8, 14.1, 16.2, 16.8 and 19.0 °2θ and wherein said XPRD pattern is measured using CuK_{α} radiation.

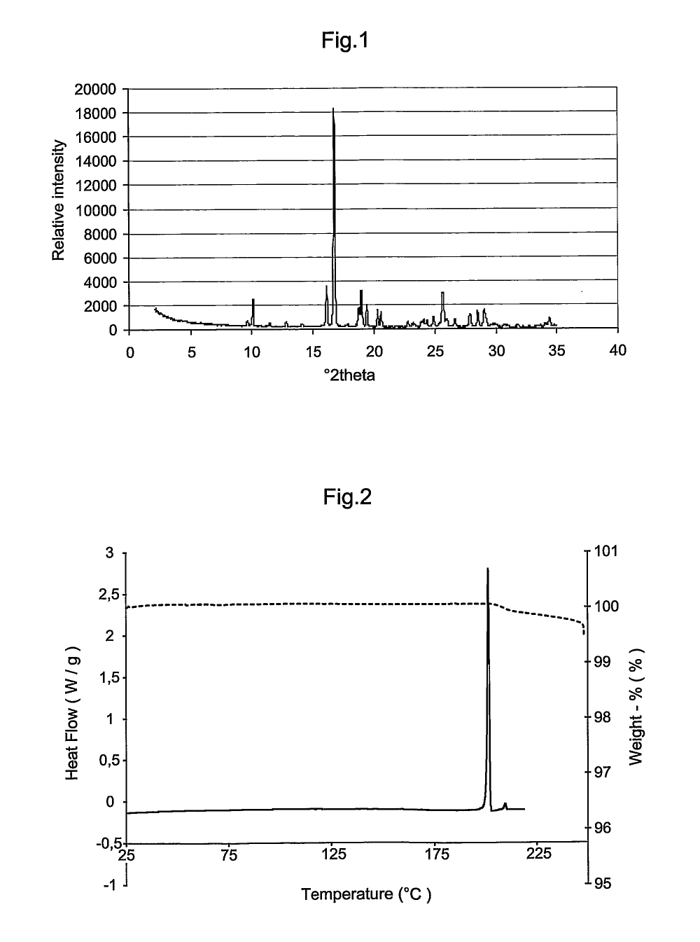

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern substantially the same as that shown in FIG. 1 and wherein said XPRD pattern is measured using CuK_{α} radiation.

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form G and characterised by having a differential scanning calorimetric (DSC) trace substantially the same as that shown in FIG. 2.

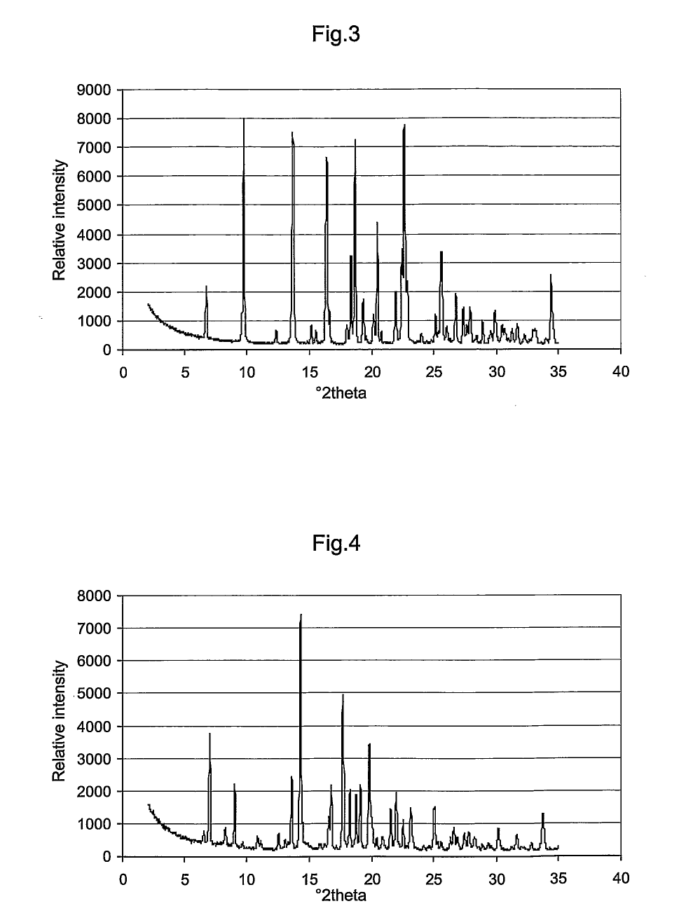

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form A and characterised by having an X-ray powder diffraction pattern comprising specific peaks at 6.8, 9.8, 13.7, 16.4, 18.4, 18.7, 20.4 and 22.6 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form A and characterised by having an X-ray powder diffraction pattern substantially the same as that shown in FIG. 3.

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form B and characterised by having an X-ray powder diffraction pattern comprising specific peaks at 6.6, 7.1, 8.3, 9.0, 13.6, 14.3, 16.8 and 17.7 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form B and characterised by having an X-ray powder diffraction pattern substantially the same as that shown in FIG. 4.

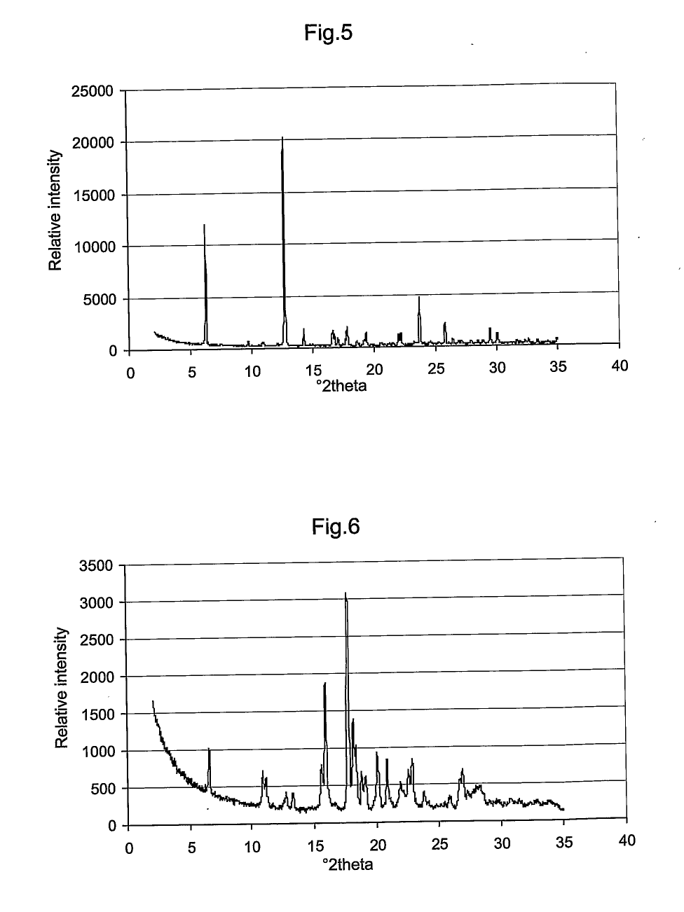

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form C and characterised by having an X-ray powder diffraction pattern comprising specific peaks at 6.3, 12.8, 14.3, 16.6, 17.8, 19.4, 22.2 and 23.7 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form C and characterised by having an X-ray powder diffraction pattern substantially the same as that shown in FIG. 5.

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form D and characterised by having an X-ray powder diffraction pattern comprising specific peaks at 6.6, 10.9, 11.2, 15.6, 15.9, 17.7, 18.2 and 18.4 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form D and characterised by having an X-ray powder diffraction pattern substantially the same as that shown in FIG. 6.

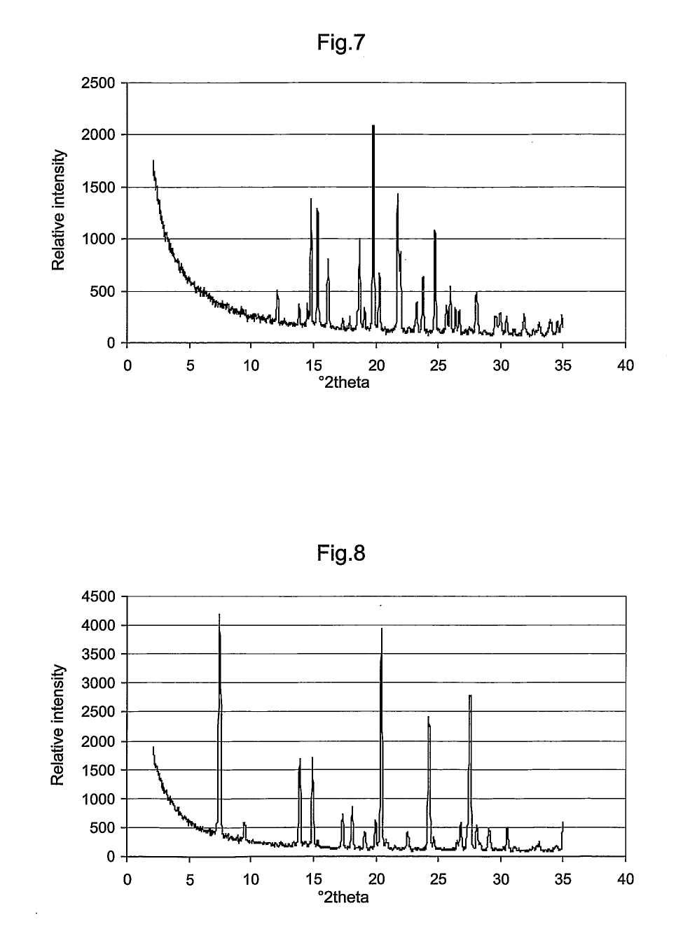

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form E and characterised by having an X-ray powder diffraction pattern comprising specific peaks at 12.1, 13.9, 14.5, 14.8, 15.3, 16.2, 18.7 and 19.8 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form E and characterised by having an X-ray powder diffraction pattern substantially the same as that shown in FIG. 7.

In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form F and characterised by having an X-ray powder diffraction pattern comprising specific peaks at 7.4, 9.5, 13.9, 14.9, 17.3, 18.1, 20.0 and 20.4 °2θ. In another embodiment, the invention provides a crystalline modification of (5S)-5-[4-(5-chloro-pyridin-2-yloxy)-piperidine-1-sulfonylmethyl]-5-methyl-imidazolidine-2,4-dione designated Form F and characterised by having an X-ray powder diffraction pattern substantially the same as that shown in FIG. 8.

It will be understood that the relative intensities of peaks in an X-ray powder diffraction (XPRD) pattern may vary according to the orientation of the sample under test and on the type and setting of the instrument used, so that the intensities in the XPRD traces included herein are to such extent illustrative and are not intended to be used for absolute comparisons.

The crystalline modifications or Forms of the invention are preferably substantially pure, meaning that each crystalline modification or Form of the compound of formula (I) includes less than 10%, preferably less than 5%, preferably less than 3%, preferably less than 1% by weight of impurities, including other crystalline modifications or Forms of the compound.

Thus, in one embodiment, the invention provides a substantially pure crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern comprising specific peaks at 10.1, 16.2, 16.8 and 19.0 °2θ and wherein said XPRD pattern is measured using CuK_{α} radiation.

In another embodiment, the invention provides a substantially pure crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern comprising specific peaks at 9.7, 10.1, 11.5, 12.8, 14.1, 16.2, 16.8 and 19.0 °2θ and wherein said XPRD pattern is measured using C K radiation.

In another embodiment, the invention provides a substantially pure crystalline modification of (5S)-5-[4-(5-chloro-pyridin-2-yloxy)-piperidine-1-sulfonylmethyl]-5-methyl-imidazolidine-2,4-dione designated Form G and characterised by having an X-ray powder diffraction (XPRD) pattern substantially the same as that shown in FIG. 1 and wherein said XPRD pattern is measured using CuK_{α} radiation.

Compound (I) Form G is obtained as a white crystalline powder comprising crystals exhibiting acicular habit. The material is essentially 100% crystalline as determined by X-ray powder diffraction measurements. The crystal structure was determined by single crystal X-ray diffraction. In the crystal, the molecules are packed in an orthorhombic space group (P2₁2₁2₁). There are 4 molecules in the asymmetric unit cell (a=10.510 Å, b=11.169 Å, c=15.560 Å). The close packing, resulting in a lack of internal space, is manifested in a relatively high density of 1.46 g/mL.

The simulated X-ray powder diffraction pattern of compound (I) Form G calculated using the single crystal X-ray diffraction data agrees well with the experimentally determined pattern shown in FIG. 1. The positions of the diffracted peaks have a very close match and the differences in relative peak intensities are attributable to preferred orientation effects.

When prepared according to the processes disclosed in WO 02/074767, (5S)-5-[4-(5-chloro-pyridin-2-yloxy)-piperidine-1-sulfonylmethyl]-5-methyl-imidazolidine-2,4-dione is obtained in amorphous phase or Form A or Form C or a mixture thereof.

A melting point for compound (I) Form A has not been observed since upon heating it transforms into Form B at about 175° C.

Compound (I) Form B is produced by solid state transitions when Form A is heated to about 175° C. Form B melts at about 207° C. and may then recrystallise to Form C and subsequently melt again at about 210° C.

Compound (I) Form C melts at about 210° C.

Compound (I) Form D is produced when compound (I) is prepared by crystallisation from a melt. For example, Form D is produced by melting Form B (starting with Form A at room temperature) at the melting temperature of Form B; then quench cooling to room temperature yielding amorphous material; and then heating again at 5°/min. During the heating, this amorphous material goes through the glass transition temperature and then subsequently recrystallises as Form D. Form D melts at about 209° C.

Compound (I) Form E is produced when Form C is slurried in water at pH 3, for example, at ambient temperature for several days. Like Form A, Form E undergoes a thermal transformation at about 175° C., probably to Form B.

Compound (I) Form F is produced when Form A or Form C is slurried in ethanol, for example, at ambient temperature for several days. Like Form A and Form E, Form F undergoes a thermal transformation at about 175° C., probably to Form B.

Compound (I) Form G is reproducibly produced when (5S)-5-[4-(5-chloro-pyridin-2-yloxy)-piperidine-1-sulfonylmethyl]-5-methyl-imidazolidine-2,4-dione is recrystallised from aqueous ethanol or from aqueous industrial methylated spirits. Form G melts at about 201° C. and, depending on the conditions used, for example, the rate of heating, may subsequently partly or completely recrystallise to Form C which then re-melts at about 210° C.

When any of compound (I) Forms A to G are heated, no solvent loss nor any other thermal event is observed prior to melting, except for possible solid state transformations, such as those outlined above, that may occur at about 175° C. Thus, each of Forms A to G is thermally stable.

The relative thermodynamic stabilities of compound (I) Forms A to G were evaluated in suspension experiments where mixtures of Forms A to G were co-incubated in water for 5 days at temperatures ranging from 5 to 40° C. In all cases, X-ray powder diffraction studies (XRPD) on the resulting precipitates demonstrated complete conversion into Form G. The same result was observed following incubation of suspensions of Form A in various organic solvents (ethanol, methanol, 1-propanol, 2-propanol, acetone or ethyl acetate). Based upon these results, it can be concluded that compound (I) Form G is the thermodynamically most stable of the seven crystalline modifications in the temperature range investigated.

Using the procedures disclosed herein, compound (I) Form G can be reproducibly manufactured following small, intermediate or large scale synthesis.

As could be anticipated from the single crystal X-ray structure determination of compound (I) Form G, which showed virtually no internal space for solvent molecules, humidity sorption measurements using gravimetrical vapour sorption (GVS) showed that the material has almost no humidity uptake even at high relative humidities (<0.05% humidity uptake at 80% RH). The material is thus advantageously classified as non-hygroscopic according to the criteria defined in the European Pharmacopoeia.

Compound (I) Form G has excellent and highly advantageous solid-state properties. It is crystalline, non-hygroscopic, and is thermally stable below 200° C., showing neither solvent loss nor any other thermal event prior to melting (see DSC and TGA traces, FIG. 2). Form G is also the thermodynamically most stable of the seven known crystalline modifications of compound (I).

The solid-state stability of compound (I) Form G was studied under three sets of conditions: at 25° C./desiccated; at 25° C./60% RH; and at 40° C./75% RH. Samples were examined after 2, 4, 8 and 12 weeks and chemical and physical stability was evaluated. It was concluded that the material was chemically and physically stable under all storage conditions since no changes were observed in the content of (5S)-5-[4-(5-chloro-pyridin-2-yloxy)-piperidine-1-sulfonylmethyl]-5-methyl-imidazolidine-2,4-dione relative to any possible degradation products (gradient RPLC); in the Form of compound (I) (XRPD); in the morphology of compound (I) (SEM); in the solvent content (TGA); or in the melting behaviour (DSC). Hence compound (I) Form G is considered to have excellent and advantageous chemical and physical stability in the solid state under pharmaceutically relevant storage conditions.

In one aspect, the present invention provides (5S)-5-[4-(5-chloro-pyridin-2-yloxy)-piperidine-1-sulfonylmethyl]-5-methyl-imidazolidine-2,4-dione Form G.

In a further aspect, the present invention provides processes for the preparation of (5S)-5-[4-(5-chloro-pyridin-2-yloxy)-piperidine-1-sulfonylmethyl]-5-methyl-imidazolidine-2,4-dione Form G. Thus, in one aspect, the invention provides a process for the preparation of (5S)-5-[4-(5-chloro-pyridin-2-yloxy)-piperidine-1-sulfonylmethyl]-5-methyl-imidazolidine-2,4-dione Form G that involves crystallisation or recrystallisation from aqueous ethanol. In another aspect, the invention provides a process for the preparation of (5S)-5-[4-(5-chloro-pyridin-2-yloxy)-piperidine-1-sulfonylmethyl]-5-methyl-imidazolidine-2,4-dione Form G that involves crystallisation or recrystallisation from aqueous industrial methylated spirits.

In another aspect, the invention provides a process for the preparation of (5S)-5-[4-(5-chloro-pyridin-2-yloxy)-piperidine-1-sulfonylmethyl]-5-methyl-imidazolidine-2,4-dione Form G that involves the following steps:
i) adding (5S)-5-[4-(5-chloro-pyridin-2-yloxy)-piperidine-1-sulfonylmethyl]-5-methyl-imidazolidine-2,4-dione to a 2:1 mixture of industrial methylated spirits (IMS):water;
ii) heating the mixture to reflux to obtain a solution;
iii) filtering the hot solution;
iv) heating the filtrate to reflux and then allowing it to cool to about 20° C. at a rate of about 0.5° C./minute;
v) collecting and drying the (5S)-5-[4-(5-chloro-pyridin-2-yloxy)-piperidine-1-sulfonylmethyl]-5-methyl-imidazolidine-2,4-dione Form G.

In a further aspect, the present invention provides (5S)-5-[4-(5-chloro-pyridin-2-yloxy)-piperidine-1-sulfonylmethyl]-5-methyl-imidazolidine-2,4-dione Form G for use in therapy.

In a further aspect, the present invention provides a crystalline modification of (5S)-5-[4-(5-chloro-pyridin-2-yloxy)-piperidine-1-sulfonylmethyl]-5-methyl-imidazolidine-2,4-dione for use in the manufacture of a medicament for the treatment or prophylaxis of diseases or conditions in which inhibition of MMP activity is beneficial.

In a further aspect, the present invention provides (5S)-5-[4-(5-chloro-pyridin-2-yloxy)-piperidine-1-sulfonylmethyl]-5-methyl-imidazolidine-2,4-dione Form G for use in the manufacture of a medicament for the treatment or prophylaxis of diseases or conditions in which inhibition of MMP activity is beneficial.

In a further aspect, the present invention provides a method of treatment or prophylaxis of a disease or condition mediated by MMP activity comprising administering to a patient in need thereof a therapeutically effective amount of a crystalline modification of (5S)-5-[4-(5-chloro-pyridin-2-yloxy)-piperidine-1-sulfonylmethyl]-5-methyl-imidazolidine-2,4-dione.

In a further aspect, the present invention provides a method of treatment or prophylaxis of a disease or condition mediated by MMP activity comprising administering to a patient in need thereof a therapeutically effective amount of (5S)-5-[4-(5-chloro-pyridin-2-yloxy)-piperidine-1-sulfonylmethyl]-5-methyl-imidazolidine-2,4-dione Form G.

In particular, compound (I) is useful in the treatment of a disease or condition mediated by MMP12 and/or MMP13 and/or MMP9 and/or MMP8 and/or MMP3; especially in the treatment of a disease or condition mediated by MMP12 and/or MMP9; most especially in the treatment of a disease or condition mediated by MMP12.

In a further aspect, the present invention provides a pharmaceutical composition comprising a crystalline modification of (5S)-5-[4-(5-chloro-pyridin-2-yloxy)-piperidine-1-sulfonylmethyl]-5-methyl-imidazolidine-2,4-dione.

In a further aspect, the present invention provides a pharmaceutical composition comprising (5S)-5-[4-(5-chloro-pyridin-2-yloxy)-piperidine-1-sulfonylmethyl]-5-methyl-imidazolidine-2,4-dione Form G.

In a further aspect, the present invention provides a method of treating a disease or condition mediated by metalloproteinase activity, comprising administering to a patient in need thereof a therapeutically effective amount of a pharmaceutical composition comprising a crystalline modification of (5S)-5-[4-(5-chloro-pyridin-2-yloxy)-piperidine-1-sulfonylmethyl]-5-methyl-imidazolidine-2,4-dione.

In a further aspect, the present invention provides a method of treating a disease or condition mediated by metalloproteinase activity, comprising administering to a patient in need thereof a therapeutically effective amount of a pharmaceutical composition comprising (5S)-5-[4-(5-chloro-pyridin-2-yloxy)-piperidine-1-sulfonylmethyl]-5-methyl-imidazolidine-2,4-dione Form G.

In a further aspect, the present invention provides the use of a pharmaceutical formulation comprising a crystalline modification of (5S)-5-[4-(5-chloro-pyridin-2-yloxy)-piperidine-1-sulfonylmethyl]-5-methyl-imidazolidine-2,4-dione for the treatment of a disease or condition in which inhibition of MMP activity is beneficial.

In a further aspect, the present invention provides the use of a pharmaceutical formulation comprising (5S)-5-[4-(5-chloro-pyridin-2-yloxy)-piperidine-1-sulfonylmethyl]-5-methyl-imidazolidine-2,4-dione Form G for the treatment of a disease or condition in which inhibition of MMP activity is beneficial.

In another aspect, the invention provides the use of a compound of formula (I) Form G in the manufacture of a medicament for the treatment or prophylaxis of inflammatory diseases or conditions; and a method of treating, or reducing the risk of, inflammatory diseases or conditions which comprises administering to a person suffering from or at risk of, said disease or condition, a therapeutically effective amount of a compound of formula (I) Form G.

Compound (I) can be used in the treatment of diseases of the respiratory tract such as obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

Compound (I) can also be used in the treatment of diseases of bone and joints such as arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies.

Compound (I) can also be used in the treatment of pain and connective tissue remodelling of musculoskeletal disorders due to injury [for example, sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis).

Compound (I) can also be used in the treatment of diseases of skin such as psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.

Compound (I) can also be used in the treatment of diseases of the eye such as blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial.

Compound (I) can also be used in the treatment of diseases of the gastrointestinal tract such as glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, non-inflammatory diarrhea, and food-related allergies which may have effects remote from the gut (for example, migraine, rhinitis or eczema).

Compound (I) can also be used in the treatment of diseases of the cardiovascular system such as atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins.

Compound (I) can also be used in oncology such as in the treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.

In particular, compound (I) may be used in the treatment of adult respiratory distress syndrome (ARDS), cystic fibrosis, pulmonary emphysema, chronic obstructive pulmonary disease (COPD), pulmonary hypertension, asthma, rhinitis, ischemia-reperfusion injury, rheumatoid arthritis, osteoarthritis, cancer, atherosclerosis and gastric mucosal injury.

More particularly, compound (I) may be used in the treatment of chronic obstructive pulmonary disease (COPD), asthma and rhinitis.

Even more particularly, compound (I) may be used in the treatment of chronic obstructive pulmonary disease (COPD).

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dose of the compound to be administered will depend on the disease being treated, the severity of the disease, the mode of administration, the age, weight and sex of the patient. Such factors may be determined by the attending physician. However, in general, satisfactory results are obtained when the compounds are administered to a human at a daily dosage of between 0.1 mg/kg to 100 mg/kg (measured as the active ingredient).

The crystalline compounds of formula (I) may be used on their own, or in the form of appropriate pharmaceutical formulations comprising the compound of the invention in combination with a pharmaceutically acceptable diluent, adjuvant or carrier. Particularly preferred are compositions not containing material capable of causing an adverse reaction, for example, an allergic reaction. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I) Form G in admixture with a pharmaceutically acceptable diluent or carrier.

We also provide a method of preparation of such pharmaceutical formulations that comprises mixing the ingredients.

The compounds may be administered topically, for example, to the lungs and/or the airways, in the form of solutions, suspensions, HFA aerosols or dry powder formulations, for example, formulations in the inhaler device known as the Turbuhaler®; or systemically, for example, by oral administration in the form of tablets, pills, capsules, syrups, powders, or granules; or by parenteral (including intraperitoneal, intravenous, subcutaneous or intramuscular injection) administration, for example, in the form of sterile parenteral solutions or suspensions; or by rectal administration, for example, in the form of suppositories.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound with a carrier substance, for example, a mono-, di- or polysaccharide, a sugar alcohol, or an other polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres that break up during the inhalation procedure. This spheronized powder may be filled into the drug reservoir of a multidose inhaler, for example, that known as the Turbuhaler® in which a dosing unit meters the desired dose which is then inhaled by the patient. With this system the active compound, with or without a carrier substance, is delivered to the patient.

For oral administration the active compound may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum, titanium dioxide, and the like. Alternatively, the tablet may be coated with a suitable polymer dissolved in a readily volatile organic solvent.

For the preparation of soft gelatine capsules, the compound may be admixed with, for example, a vegetable oil or polyethylene glycol. Hard gelatine capsules may contain granules of the compound using either the above mentioned excipients for tablets. Also liquid or semisolid formulations of the drug may be filled into hard gelatine capsules.

Liquid preparations for oral application may be in the form of syrups or suspensions, for example, solutions containing the compound, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

In a further aspect of the invention we provide novel processes for the synthesis of compound (I). In particular, novel processes for the synthesis of crystalline modifications of compound (I) are disclosed. In particular, novel processes for the synthesis of compound (I) Form G are disclosed.

A preferred process for the synthesis of compound (I) is shown in Scheme 2.

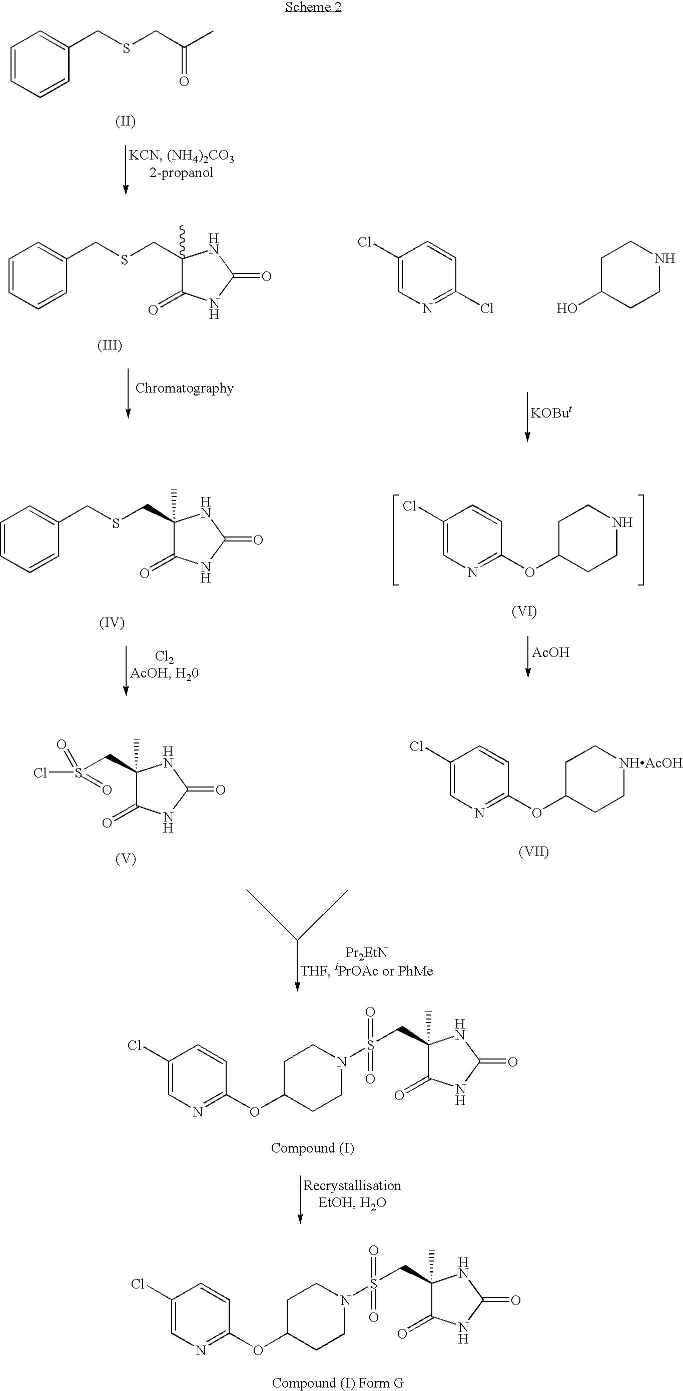

In Scheme 2, the sulphur moiety in compounds (II), (III) and (IV) is protected as an S-benzyl derivative. The skilled man will readily appreciate that other suitable protecting groups such as t-butyl may alternatively be used. Thus, whilst for convenience subsequent reactions are shown using S-benzyl protected compounds, it is to be understood that suitable alternative protecting groups such as t-butyl may also be used.

5-Chloro-2-(piperidin-4-yloxy)-pyridine (VI) is a waxy solid (m.p. about 43° C.) and as such, crystallisation and isolation of this material, particularly on a large scale, is not ideal. The preparation of a salt such as the acetate salt (VII) allows the compound to be isolated as a solid that is more conveniently handled. Salts other than the acetate may also be used. Such salts include the phosphate, mono-hydrochloride, di-hydrochloride, trimethylacetate, tartrate, citrate, fumarate, maleate, benzoate, mono-hydrobromide, di-hydrobromide, carbonate and hemi-carbonate. The carbonate salts are particularly useful since they are thermally labile so that the free base can be liberated in situ simply by warming.

Thus, in one aspect we disclose an improved procedure for the isolation and handling of 5-chloro-2-(piperidin-4-yloxy)-pyridine (VI) which involves the intermediacy of 5-chloro-2-(piperidin-4-yloxy)-pyridine acetate salt (VII).

In another aspect, we disclose novel salts of 5-chloro-2-(piperidin-4-yloxy)-pyridine (VI) useful as intermediates in the preparation of compound (I).

In a preferred process, the synthesis of 5-chloro-2-(piperidin-4-yloxy)-pyridine acetate (VII) is advantageously carried out in a solvent such as toluene. The use of toluene as the reaction solvent allows the reaction of 2,5-dichloropyridine with 4-hydroxypiperidine, subsequent aqueous washes and salt formation to be performed in the same reaction vessel without the need for isolation of the intermediate free base. As water is a critical parameter in this reaction (and 4-hydroxypiperidine is hygroscopic) the use of toluene to azeotropically remove water prior to starting the reaction represents a significant improvement and allows consistent yields to be isolated, even on a multi-kilogram scale.

The preparation of (RS)-5-methyl-5-{[(phenylmethyl)thio]methyl}imidazolidine-2,4-dione (III) from benzylthioacetone (II) is described in WO 02/074767. Compared to the conditions described therein we now disclose an improved process wherein the organic solvent is changed from ethanol to 2-propanol and the amount of potassium cyanide used is reduced from 2 equivalents to about 1.00 to 1.02 equivalents. In this way, the potassium cyanide is essentially completely consumed in the reaction and the need to handle and dispose of solutions containing large amounts of unreacted potassium cyanide is avoided. We further disclose that it is particularly advantageous to reduce the amount of ammonium carbonate used from about 5 equivalents to about 1.1 to 1.25 equivalents. In this way the maximum operating pressure is reduced from about 9 barg to about 1.5 to 2.5 barg, a significant safety advantage, particularly for larger scale work. Using these revised parameters, the synthesis of the (RS)-5-methyl-5-{[(phenylmethyl)thio]methyl}-imidazolidine-2,4-dione (III) has been routinely run on a multi-kilogram scale.

Thus, in another aspect we disclose improved conditions for the preparation of the (RS)-5-methyl-5-{[(phenylmethyl)thio]methyl}imidazolidine-2,4-dione (III) from benzylthioacetone (II). These improved conditions are particularly advantageous for large scale preparations.

As described in WO 02/074767, the separation of (RS)-5-methyl-5-{[(phenylmethyl)thio]methyl}imidazolidine-2,4-dione (III) into the constituent enantiomers is conveniently achieved by chiral HPLC using a Chiralpak AD column as the stationary phase and methanol as eluent. As an alternative that is particularly convenient for larger scale work, we now disclose a process wherein the chiral separation is carried out under essentially the same conditions but using simulated moving bed (SMB) chromatography. In this way, (S)-5-methyl-5-{[(phenylmethyl)thio]methyl}imidazolidine-2,4-dione (IV) may be obtained on a multi-kilogram scale. The unprotected thiol, (RS)-5-methyl-5-thiomethyl-imidazolidine-2,4-dione, is surprisingly stable and may also be is conveniently resolved by chiral HPLC using a Chiralpak AD column as the stationary phase and isohexane/ethanol/diethylamine as the mobile phase.

As an alternative to chiral chromatography, other routes to the chiral imidazolidine-2,4-dione (IV) are disclosed.

The use of (S)-α-methylbenzylamine in the resolution of certain hydantoin derivatives has been previously disclosed (WO 92/08702). We have now found that racemic (RS)-5-methyl-5-{[(phenylmethyl)thio]methyl}imidazolidine-2,4-dione (III) may be resolved by crystallisation from a suitable solvent in the presence of a chiral amine and of a base such as sodium hydroxide. Examples of chiral pool amines include (1S)-(−)-α-methylbenzylamine, (1R)-(+)-α-methylbenzylamine, L-tyrosinamide, (1S)-(−)-α-(1-naphthyl)ethylamine, (1R)-(+)-α-(1-naphthyl)ethylamine, L-(−)-cinchonidine, D-(+)-cinchonine, (−)-quinine, (+)-β-quinidine, (1R,2S)-(−)-ephedrine, (2R)-(−)-2-amino-1-butanol, (2R)-1-amino-2-propanol (D-alaninol), (1R,2S)-(−)-2-amino-1,2-diphenylethanol, N-methyl-D-(−)-glucamine, (2S)-(+)-2-phenylglycinol, norephedrine, (−)-brucine, (−)-strychnine, (+)-yohimbine, (1S,2S)-(+)-threo-2-amino-1-(p-nitrophenyl)-1,3-propanediol, (L)-(+)-threo-2-amino-1-phenyl-1,3-propanediol, cis-myrtanylamine, (1R,2R)-(−)-1,2-diaminocyclohexane and (2R)-(−)-2-amino-2-phenylethanol.

In a preferred procedure, the chiral amine is (1S)-(−)-α-methylbenzylamine.

Thus, in one aspect, we disclose a process for the resolution of racemic (RS)-5-methyl-5-{[(phenylmethyl)thio]methyl}imidazolidine-2,4-dione (III) using (1S)-α-methylbenzylamine.

In a preferred procedure, the chiral amine is (1S)-(−)-α-methylbenzylamine (1.0 to 2.0 equivalents), the base is sodium hydroxide (0.4 to 0.6 equivalents) and the solvent is water (4 to 8 vols). Crystallisation then affords (5S)-5-benzylthiomethyl-5-methyl-imidazolidine-2,4-dione (S)-α-methyl benzylamine of high enantiomeric purity, generally >95%. Further conversion of this material into (5S)-5-methyl-5-{[(phenylmethyl)thio]methyl}-imidazolidine-2,4-dione (IV) may be effected under standard conditions, for example, using 2N hydrochloric acid, or simply by crystallisation from a variety of suitable solvents including isopropyl acetate, methyl isobutyl ketone (MIBK), toluene, t-butylmethyl ether (TBME), and combinations of such solvents. The conversion of (5S)-5-benzylthiomethyl-5-methyl-imidazolidine-2,4-dione (S)-α-methyl benzylamine into (IV) may also be effected simply by slurrying the solid in suitable, hot solvent such as cyclohexane, dibutylether or water. Thus, the act of warming the co-crystal in solution, or as a slurry, causes the liberation of the free (5S)-5-methyl-5-{[(phenylmethyl)thio]methyl}-imidazolidine-2,4-dione (IV) which then crystallises on cooling. Further chiral enhancement is observed when using either of these methods to liberate (IV).

In another aspect, racemic 2-amino-3-benzylthio-2-methylpropionamide (VIII)

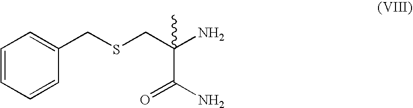

may be resolved by crystallisation from a suitable solvent in the presence of a suitable chiral acid. Examples of chiral pool acids include (L)-tartaric acid, (R)-(−)-mandelic acid, dibenzoyl-(L)-tartaric acid [DBTA], di-p-toluoyl-(L)-tartaric acid [DTTA], (L)-malic acid [(2S)-(−)-2-hydroxysuccinic acid], (1S)-(+)-10-camphorsulphonic acid [(D)-CSA], (1R,3S)-(+)-camphoric acid [cis-camphoric acid], (L)-glutamic acid [(2S)-(+)-2-aminopentanedioic acid], (L)-aspartic acid [(S)-(+)-aminosuccinic acid], (L)-pyroglutamic acid [(S)-(−)-2-pyrrolidone-5-carboxylic acid], (L)-ornithine hydrochloride [(2S)-(+)-2,5-diaminopentanoic acid], (L)-histidine, (L)-lysine [(2S)-(+)-2,6-diaminohexanoic acid], (L)-arginine, N-acetyl-(L)-phenylalanine, N-acetyl-(L)-leucine, N-carbobenzyloxy-(L)-alanine [(2S)-2-benzyloxycarbonylaminopropionic acid], (−)-menthoxyacetic acid, N-acetyl-(L)-tyrosine and (2R)-(+)-2-(4-hydroxyphenoxy)propionic acid.

In one preferred process, the chiral acid is (R)-(−)-mandelic acid.

Thus, in one aspect, we disclose a process for the resolution of racemic 2-amino-3-benzylthio-2-methylpropionamide (VIII) using (R)-(−)-mandelic acid.

In one preferred process, the chiral acid is (R)-(−)-mandelic acid and the solvent is a mixture of methanol and isopropyl acetate. The crystallisation must be performed in the presence of water. In this way, (2S)-2-amino-3-benzylthio-2-methylpropionamide (R)-mandelate hemihydrate of high enantiomeric purity is obtained. The enantiomeric purity of this salt may be further enhanced by recrystallisation from a solvent such as isopropyl acetate.

In another preferred procedure, the chiral acid is L-tartaric acid.

Thus, in one aspect, we disclose a process for the resolution of racemic 2-amino-3-benzylthio-2-methylpropionamide (VIII) using L-tartaric acid.

In another preferred procedure, the chiral acid is L-tartaric acid and the solvent is ethanol. Recrystallisation of the resulting (2S)-2-amino-3-benzylthio-2-methylpropionamide (L)-tartrate from a suitable solvent such as a mixture of methanol and methyl isobutyl ketone then affords material of high enantiomeric purity.

The further conversion of (2S)-2-amino-3-benzylthio-2-methylpropionamide into (5S)-5-methyl-5-{[(phenylmethyl)thio]methyl}imidazolidine-2,4-dione (IV) may be achieved using methods that will be readily apparent to the man skilled in the art. See, for example, Tetrahedron Asymm., 2001, 12, 101; Tetrahedron, 1991, 47(12), 2133; and Chem. Ber., 1928, 1431.

In another aspect, chiral 5-methyl-5-{[(phenylmethyl)thio]methyl}imidazolidine-2,4-dione (IV) may be prepared via biocatalytic (enzymatic) resolution of a suitable racemic precursor molecule. Certain possible routes are outlined in Scheme 3.

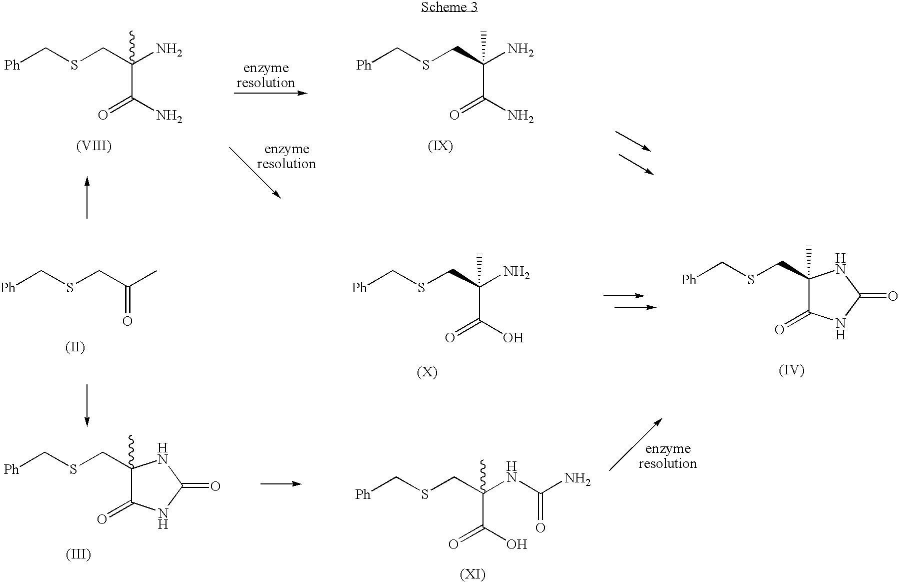

As indicated in Scheme 3, either (S)-2-amino-3-benzylsulfanyl-2-methylpropionamide (IX) or (S)-2-amino-3-benzylsulfanyl-2-methylpropionic acid (X) may serve as a suitable precursor to the desired chiral hydantoin (IV).

The biocatalytic resolution of the racemic amino amide (VIII) requires the use of an amidase that is capable of accepting this rather sterically hindered type of substrate. The use of the amidases Mycobacterium neoaurum ATCC 25795 or Ochrobactrum anthropi NCIMB 40321 for the resolution of C^{α}-tetrasubstituted α-amino amides is described in Tetrahedron, 2001, 57, 6567-6577. Mycobacterium neoaurum proved to be a suitable amidase for the resolution of the particular amino amide (VIII), but Ochrobactrum anthropi surprisingly gave racemic hydrolysis. Other amidases that could be successfully employed in the resolution of the amino amide (VIII) include Rhodococcus erthoplis and Pseudomonas fluorescens AL45. The resolution of amino amide (VIII) using Pseudomonas fluorescens AL45 is disclosed in WO 2005/123932.

As shown in Scheme 4, the stereochemical outcomes of these biocatalytic resolutions can conveniently be controlled by choice of the appropriate amidase. Typical specific procedures for the biocatalytic resolution of the racemic amino amide (VIII) are given in the Examples section of the present application and such processes represent specific aspects of the present invention.

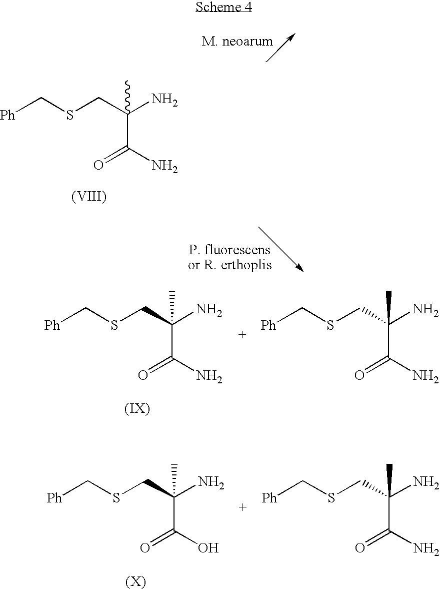

In an alternative biocatalytic approach, the racemic α-ureido acid (XI), prepared by hydrolysis of racemic hydantoin (III) or from the corresponding racemic amino acid, is subjected to a hydantoinase-catalysed ring closure (Scheme 5). Suitable hydantoinases include Roche Hydantoinase 1 and Hydantoinase 2.

In one aspect, we disclose a process for the preparation of (S)-5-methyl-5-{[(phenylmethyl)thio]methyl}imidazolidine-2,4-dione (IV), useful as an intermediate in the synthesis of (5S)-5-[4-(5-chloro-pyridin-2-yloxy)-piperidine-1-sulfonylmethyl]-5-methyl-imidazolidine-2,4-dione, which comprises use of a hydantoinase enzyme to effect ring closure of (RS)-3-benzylsulfanyl-2-methyl-2-ureido-propionic acid (XI). In a further aspect, the hydantoinase enzyme is Roche Hydantoinase 1 or Hydantoinase 2.

Alternative biocatalytic methods for the resolution of α-ureido acids are described in EP 0 175 312 (Kanegafuchi) and WO 03/106689 (Kaneka).

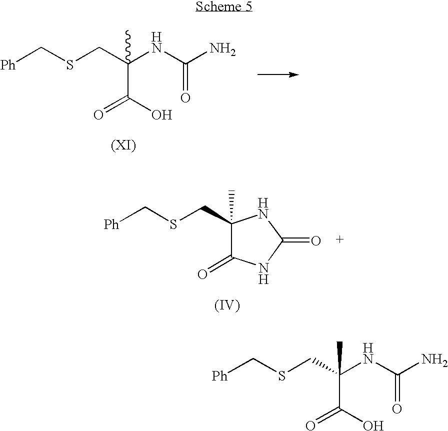

In an alternative biocatalytic approach (Scheme 6), the racemate of the amino acid (X) is converted into the corresponding trifluoroacetyl protected amino acid (XII) which is then subjected to amino acid acylase catalysed hydrolysis. Suitable amino acid acylases include Aspergillus sp., L-Hog kidney acylase and L-acylase from Penicillium sp. Other suitable acylases will be readily apparent to the man skilled in the art.

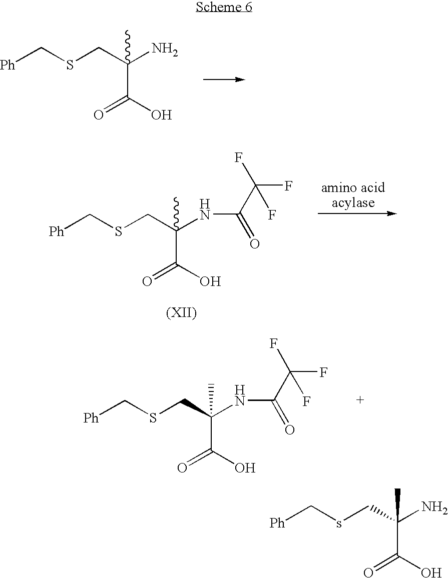

Surprisingly, more traditional substrates such as the N-acetyl or N-chloroacetyl amides corresponding to compound (XII) failed to show any reaction with L-amino acid acylase. The man skilled in the art will readily appreciate that the trifluoroacetyl amide (XII) could be replaced by other activated amides and that the selectivity of the resolution could be reversed by substituting a D-amino acid acylase, thereby facilitating the direct crystallisation of the (S)-amino acid from the reaction mixture.

In one aspect, we provide a process for the preparation of (R)- or (S)-2-amino-3-benzylsulfanyl-2-methyl-propionic acid, useful as intermediates in the synthesis of (R)- or (S)-5-methyl-5-{[(phenylmethyl)thio]methyl}imidazolidine-2,4-dione, which comprises treating an activated amide of (RS)-2-amino-3-benzylsulfanyl-2-methyl-propionic acid with a suitable acylase enzyme. In one particular aspect, the activated amide is a trifluoroacetyl amide.

In another aspect, chiral 5-methyl-5-{[(phenylmethyl)thio]methyl}imidazolidine-2,4-dione (IV) may be prepared via biocatalytic (enzymatic) desymmetrisation of a suitable achiral (meso) precursor molecule. The enzymatic transformations which have been described above are all resolutions whereby the theoretical maximum yield of the desired stereoisomer is 50%. In contrast, desymmetrisation of simple prochiral (meso) compounds can in theory yield 100% of the desired stereoisomer. Certain possible routes are outlined in Scheme 7.

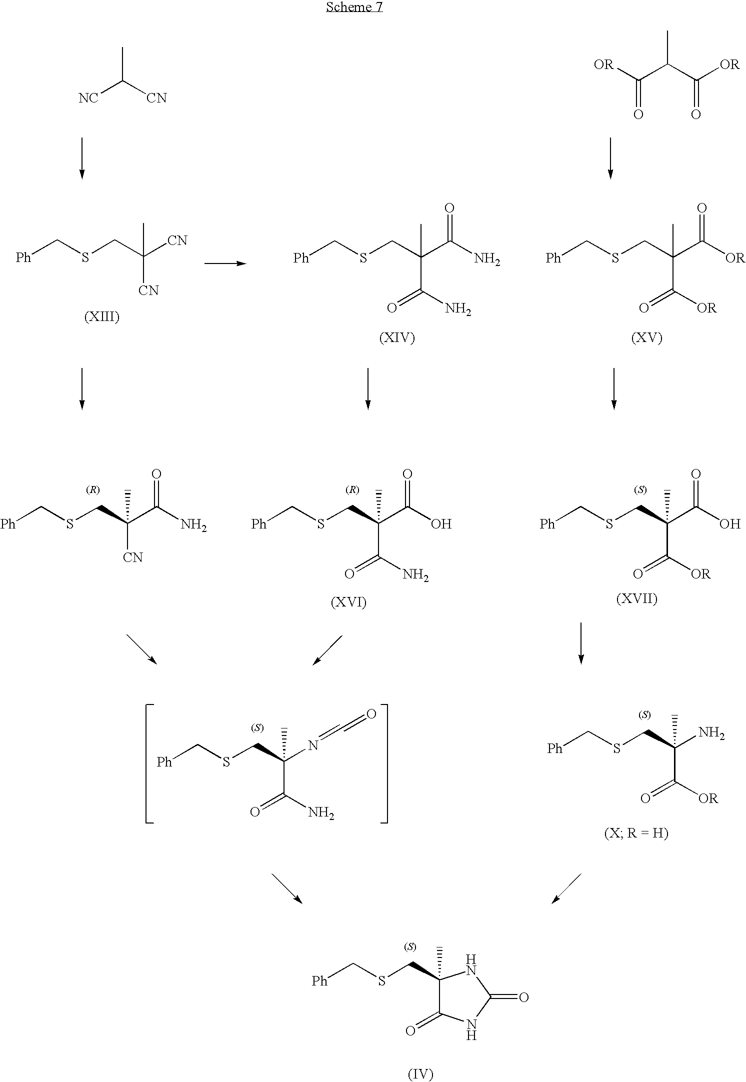

Thus, suitable meso-precursors such as the nitrile (XIII), the amide (XIV) or an ester (XV) may be desymmetrised using a suitable enzyme thereby affording the chiral hydantoin precursors shown above. Suitable R groups for the esters (XV) include C1 to 4 alkyl.

The required meso-precursors may be prepared using methods analogous to those described in the literature. See, for example, J. Org. Chem., 1995, 60(17), 5487; J. Chem. Soc., Perkin Trans. 1, 1991, 4, 2589; Synth. Comm., 2001, 1323; and Inorg. Chem., 2003, 42(9), 2950. The synthesis of specific meso-precursors is disclosed in the Examples section.

Potential enzymes for the desymmetrisation of the meso-nitrile (XIII) are described in, for example, Tetrahedron Asym., 2004, 15, 2817; Tetrahedron Asym., 2001, 12, 3367; Tetrahedron Asym., 1993, 4, 1081; and J. Org. Chem., 2003, 68, 2479.

Desymmetrisation of the meso-amide (XIV) was achieved using Rhodococcus erthoplis amidase. The resultant chiral acid amide (XVI) was then further transformed in a single pot sequence to afford the chiral hydantoin (IV) with excellent ee.

Desymmetrisation of a meso-S-t-butyl methyl ester using Pig Liver Esterase has been previously disclosed (J. Org. Chem., 2003, 68(13), 5403).

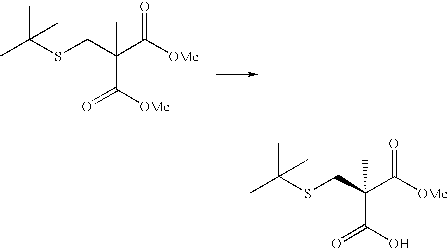

It has now been shown that the meso-S-benzyl ethyl ester (XV, R=Et) is also a substrate for this enzyme. Desymmetrisation proceeded in line with the literature precedent, affording (after Curtius rearrangement of the initially formed acid ester (XVII) and subsequent ester hydrolysis), the (R)-amino acid (X) in 60-80% ee.

It has been further established that similar desymmetrisation transformations may be achieved using representatives of two different enzyme classes, namely, Bacillus licheniformis protease and an amino acid acylase. In the case of desymmetrisation using Bacillus licheniformis protease the transformation proceeded with the reverse stereoselectivity to afford the (S)-ester acid. This (S)-ester/acid was further converted into the corresponding amino acid, the absolute configuration and chiral purity of which were determined by comparison with an authentic sample. Following methods well known in the literature (see, for example, Chem. Rev., 1950, 46, 403), the amino acid into the (S)-5-methyl-5-{[(phenylmethyl)thio]methyl}imidazolidine-2,4-dione (IV). Thus, in a further aspect, we disclose a process for the synthesis of (S)-5-methyl-5-{[(phenylmethyl)thio]methyl}imidazolidine-2,4-dione (IV), useful as an intermediate in the synthesis of compound (I), which comprises enzymatic desymmetrisation of a meso-nitrile (XIII), or a meso-amide (XIV) or a meso-ester (XV). In a particular aspect, we disclose a process wherein the meso-amide (XIV) is desymmetrised using a suitable amidase enzyme. In another particular aspect, the amidase is Rhodococcus erthoplis amidase.

In the above biocatalytic resolutions, the enzyme may, where appropriate, be used as such or in an immobilised (supported) form.

In another aspect, chiral 5-methyl-5-{[(phenylmethyl)thio]methyl}imidazolidine-2,4-dione (IV) may be prepared via asymmetric synthesis.

The asymmetric Strecker reaction is an important method for the synthesis of α,α-dialkyl amino acids. (R)-Phenylglycinol is a typical chiral auxiliary for use in such reactions (Tetrahedron, 2001, 57, 6383-6397). Thus, condensation of benzylthioacetone (II) with (R)-(−)-phenylglycinol affords the oxazolidine (XVIII) as a mixture of diastereomers. Reaction of the oxazolidine (XVIII) with trimethylsilylcyanide then gives the amino nitrile (XIX) as mixture of diastereomers in a ratio of 85:15. Recrystallisation of this mixture from a suitable solvent such as iso-hexane increases the diastereomeric ratio of compound (XIX) to greater than 99:1 (Scheme 8):

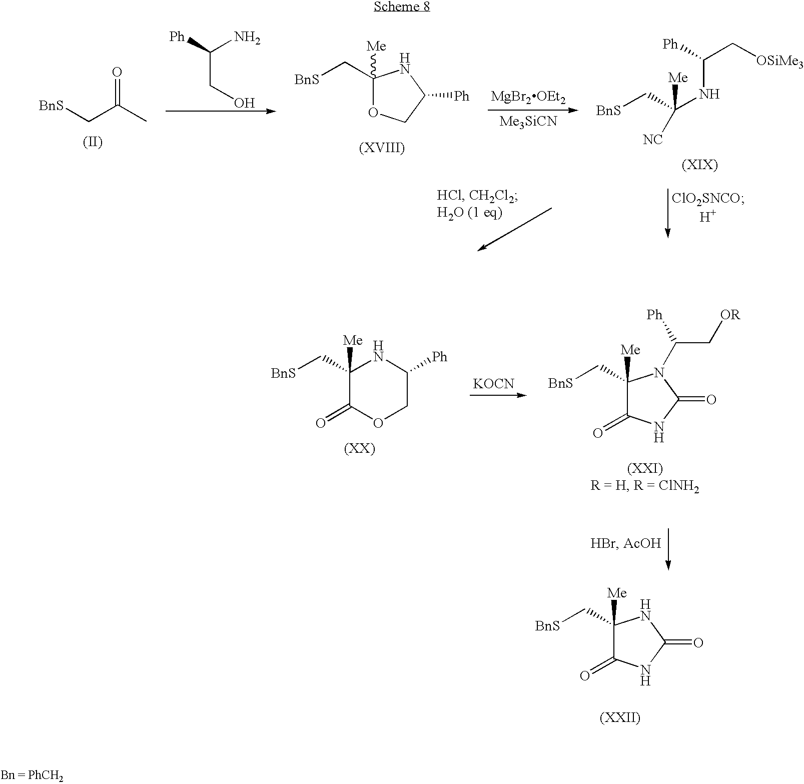

Treatment of the amino nitrile (XIX) with one equivalent of water in the presence of hydrogen chloride gas then gives the lactone (XX). Reaction with potassium cyanate followed by removal of the side chain using hydrogen bromide in acetic acid then affords the chiral hydantoin (XXII).

Alternatively, treatment of the amino nitrile (XIX) with chlorosulfonyl isocyanate gave a mixture comprising the hydantoins (XXI; R═H) and (XXI; R═CONH₂), which mixture when treated with hydrogen bromide in acetic acid gave the chiral (R)-hydantoin (XXII).

By using (S)-phenylglycinol as the chiral auxiliary, the enantiomeric (S)-hydantoin is obtained.

In one aspect, we disclose a process for the synthesis of (R)- or (S)-5-methyl-5-{[(phenylmethyl)thio]methyl}imidazolidine-2,4-dione which comprises ring opening of a chiral auxiliary labelled oxazolidine (XVIII).

We further disclose the synthesis of chiral hydantoins using asymmetric phase transfer catalysis. Catalytic asymmetric processes are especially attractive in this regard since they allow for the use of substoichiometric amounts of the chiral control element, which is often the most expensive reagent in the process. Chiral phase-transfer catalysis offers further advantages because it typically involves mild conditions, simple reaction procedures, safe and inexpensive reagents and solvents, the use of organocatalysts (catalysts without metals), and the possibility of conducting reactions on either a small or large scale. However, the construction of compounds containing quaternary stereogenic centres (carbons with four different non-hydrogen groups) by catalytic enantioselective processes continues to be challenging. A suitable route which makes possible the construction of such a quaternary stereogenic centre is outlined in Scheme 9.

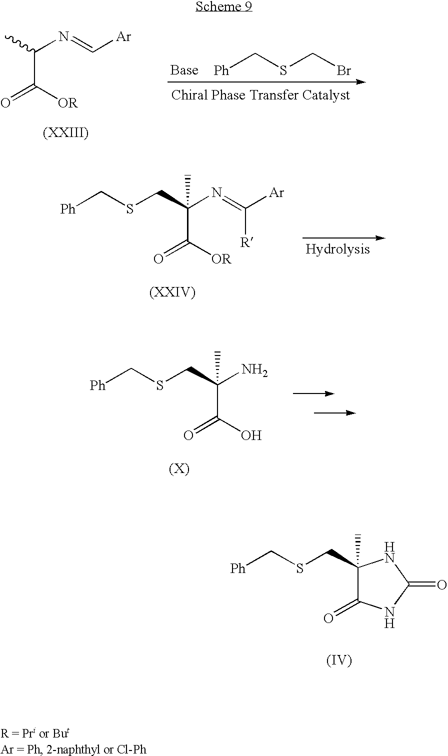

In an initial step, t-butyl (DL)-alaninate or isopropyl (DL)-alaninate is condensed with a suitable carbonyl derivative such as benzaldehyde, chlorobenzaldehyde or 2-naphthaldehyde to give an imine ester (XXIII). Preferably the t-butyl ester is used. This imine is then alkylated with bromomethylsulfanylmethylbenzene in the presence of a suitable base and a suitable chiral phase transfer catalyst to give the imine (XXIV). Suitable bases include, for example, potassium hydroxide, sodium hydride, caesium; hydroxide and rubidium hydroxide. Suitable phase transfer catalysts include, for example, (−)-O-allyl-N-(9-anthracenylmethyl)cinchonidinium bromide and (+)-O-allyl-N-(9-anthracenylmethyl)cinchonidinium bromide. Selection of the correct pseudo-enantiomeric (antipodal) phase transfer catalyst allows the absolute stereochemistry of the resultant imine (XXIV) to be controlled. Other suitable chiral phase transfer catalysts will be readily apparent to the man skilled in the art.

Hydrolysis of the imine (XXIV) then yields the α-amino acid (X). By choice of the correct reagents and the correct reaction conditions, the chiral α-amino acid (X), or the enantiomer thereof, is obtained in high enantiomeric purity. In a particular aspect, the specific procedures disclosed in the accompanying Examples are specifically claimed.

In one aspect, we provide a process for the preparation of (R)- or (S)-2-amino-3-benzylsulfanyl-2-methyl-propionic acid, useful as intermediates in the synthesis of (R)- or (S)-5-methyl-5-{[(phenylmethyl)thio]methyl}imidazolidine-2,4-dione, which comprises alkylation of an imine ester (XXIII) in the presence of a suitable chiral phase transfer catalyst. In one particular aspect, the phase transfer catalyst is (−)-O-allyl-N-(9-anthracenylmethyl)cinchonidinium bromide or (+)-O-allyl-N-(9-anthracenylmethyl)-cinchonidinium bromide. The skilled man will readily appreciate that in the above process the sulphur atom could alternatively be protected by a group other than benzyl.

The chiral α-amino acid (X) may then be further converted into the chiral hydantoin (IV) using literature procedures. See, for example, Chem. Rev., 1950, 46, 403.

The preparation of ((S)-4-methyl-2,5-dioxo-imidazolidin-4-yl)-methanesulfonyl chloride (V) from (S)-5-methyl-5-{[(phenylmethyl)thio]methyl}imidazolidine-2,4-dione (IV) by direct chlorination in aqueous acetic acid is disclosed in WO 02/074767. Novel alternative processes for the preparation of the sulfonyl chloride (V) are disclosed in a co-pending patent application, U.S. 60/782,892.

For coupling with the sulfonyl chloride (V), the piperidinyl ether acetate salt (VII) must first be reconverted into the corresponding free base (VI). This conversion may be achieved using a base such as sodium carbonate in the presence of an ester solvent such as ethyl acetate or isopropyl acetate. In a preferred process, the conversion is achieved in a biphasic system by suspending the acetate salt in toluene and using aqueous sodium is hydroxide as base. The use of toluene enables more efficient drying of the solution of the free base (VI) by azeotropic distillation. This is an important advantage since the coupling reaction of compound (VI) with the sulfonyl chloride (V) is particularly sensitive to the presence of water. In either case, the obtained solution of the free base (VI) in either isopropyl acetate or toluene is then reacted directly with the sulfonyl chloride (V) in the presence of a suitable base such as diisopropylethylamine and using tetrahydrofuran as co-solvent. In this way, compound (I) is conveniently and efficiently prepared even on a multi-kilogram scale.

In one aspect, we disclose a process for the preparation of compound (I) that involves reaction of 5-chloro-2-(piperidin-4-yloxy)-pyridine (VI) with ((S)-4-methyl-2,5-dioxo-imidazolidin-4-yl)-methanesulfonyl chloride (V) wherein compound (VI) is prepared by liberation of the free base from a corresponding salt. In a more particular aspect, compound (VI) is prepared by liberation of the free base from 5-chloro-2-(piperidin-4-yloxy)-pyridine acetate salt (VII).

No crystalline modifications of compound (I) are disclosed in WO 02/074767. We have now found that compound (I), prepared by any synthetic method, may be crystallised using aqueous ethanol or aqueous industrial methylated spirits as solvent to reproducibly afford compound (I) modification G, irrespective of the polymorphic modification of the input material.

The identification of the different polymorphic modifications and their crystallinity were investigated using the following instruments and methods:

### X-Ray Powder Diffraction (XPRD)

XRPD measurements were made using either:

i) A Scintag Inc. XDS 2000 instrument with the following parameters:
CuK_{α} (1.5418 Å)
45 kV and 30 mA
2°≦2θ≦35°
1°/min, incr. 0.03°
Rotating quartz disc
Ambient conditions

Approximately 10 mg of the test sample was placed on the sample holder and smeared out on the quartz surface using a flat Teflon bar; or

ii) A Panalytical X'Pert PRO MPD instrument with the following parameters:
CUK_{α} (1.5418 Å)
45 kV and 40 mA
2°≦2θ≦40°
4°/min, incr. 0.016°
Rotating Silicon wafer
Ambient conditions

Approximately 2 mg of the test sample was placed on the sample holder and smeared out on the silicon surface using a flat Teflon bar.

### Calorimetry (DSC)

The calorimetric response of a test sample to increasing temperature was investigated using a Q1000 Modulated Temperature Differential Scanning Calorimeter (MTDSC) (TA Instruments) using different methods, the main features being:

Normally modulated mode (“heat only”) with a ramp rate of 5° C./min (but also 1 and 20° C./min were used without modulation). The temperature range was from just below ambient to above 200° C.

Approximately 2 mg of the test sample was placed in an aluminium cups with a lid (no crimping).

### Gravimetric Analysis (TGA)

The gravimetric response of test samples to increasing temperatures was investigated using a Q500 Thermal Gravimetric Analyser (TGA) (TA Instruments) using the following parameters:

Heating rate (normally): 5° C./min

Approximately 2 to 5 mg of the test sample was placed in the cup and heated to just above 200° C.

### Humidity Interaction

The gravimetric responses of test samples to changes in humidity were investigated using either a SGA 100 (VTI Corporation) or a DVS 2 (Surface Measurement System) Gravimetrical Vapour Sorption (GVS) instrument with the following features: Dry to 90% RH and back in steps of, for example, 10% RH.

Equilibrium condition: <0.01 weight-% per 10 minutes (<0.001 weight-%/min)

Approximately 5 mg of the test sample was placed in the cup and evaluated.

### Morphology

The morphology of the test compound was investigated using a Jeol JSM-5200 Scanning Electron Microscope (SEM) using a magnification of up to 5000 times.

A few particles were sprinkled onto the sample holder with a carbon sticky tape and coated with a thin gold layer and investigated.

### General Chemical Methods

¹H NMR and ¹³C NMR spectra were recorded on a 300 MHz Varian Unity Inova or 400 MHz Varian Unity Inova instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Starting materials were commercially available unless otherwise stated. All solvents and commercial reagents were of laboratory grade and were used as received. Unless otherwise stated, operations were conducted at ambient temperature, typically 20 to 25° C.

LC analysis was performed using Agilent 1100 HPLC instruments. Various LC methods were used for product analysis.

LCMS analysis was performed using WATERS 2790 HPLC with 996 Photo Diode Array Detector and MicroMass ZMD, Single Quadrupole Mass Spectrometer with Z-spray interface.

Abbreviations:

vol eq. grams of limiting substance expressed as a volume
Ac acetyl
DCM dichloromethane

### DMF N,N-dimethylformamide

DMSO dimethyl sulfoxide
ep enantiomeric purity
eq. equivalent
IMS industrial methylated spirits
LDA lithium diisopropyl amide
MIBK methyl isobutyl ketone
RT room temperature
TBME tert-butyl methyl ether
THF tetrahydrofuran
TFA trifluoroacetic acid

### Example 1

### 5-Chloro-2-(piperidin-4-yloxy)-pyridine acetate

4-Hydroxypiperidine (12.1 g, 0.12 mol, 1.18 mol eq.) was suspended in toluene (120 mL) to give an orange suspension. The resulting mixture was heated to reflux over 15 minutes (jacket temperature 115° C.). An orange solution formed between 85-90° C. and some oiling was observed on the stirrer shaft and temperature probe. Toluene (26 mL) was removed by distillation. The reaction mixture was cooled to 20° C. over 15 minutes. A white solid precipitated at about 30 to 35° C. In a separate vessel, tert-BuOK (13.4 g, 0.12 mol, 1.18 mol eq.) was suspended in toluene (150 mL). This suspension was added to the above 4-hydroxypiperidine mixture at 20° C. A toluene line wash (11 mL) was then performed. The resulting thick suspension was heated up to 50° C. over 30 minutes with vigorous stirring. A solution of 2,5-dichloropyridine (15 g, 0.10 mol, 1 mol eq.) in toluene (45 mL), was added to the slurry at 50° C. over approximately 1 h, followed by a toluene line wash (11 mL). The reaction mixture was warmed to reflux (ca. 105-107° C.) over 70 minutes and was then heated at reflux for 2 h. The reaction mixture was cooled to ambient temperature over 30 minutes and stirred overnight. The reaction mixture was washed with water (2×75 mL) then heated to 90° C. over 1 h. A solution of glacial acetic acid (6.1 g, 0.10 mol, 1 mol eq.) in toluene (60 mL) was added to the mixture at 90° C. in one portion, followed by a toluene line wash (15 mL). After addition was complete, the solution was cooled to RT over 70 minutes. The required 5-chloro-2-(piperidin-4-yloxy)-pyridine acetate precipitated during cooling. After stirring for 1 h at RT, the suspension was filtered and the cake washed with toluene (2×75 mL). After drying in a vacuum oven at 50° C. overnight, the product was obtained in 85 to 95% yield.

¹H NMR (400 MHz, D₂O) δ 8.1 (1H, d), 7.8 (1H, dd), 6.9 (1H, d), 5.0 (1H, m), 3.4 (4H, m), 2.2 (4H, m), 1.9 (3H, s).

### Alternative Salts of 5-Chloro-2-(piperidin-4-yloxy)-pyridine

The appropriate acid (either in a solvent for mineral acids; or as a solid for organic acids; or, for the carbonate salt, CO₂ gas was bubbled into the solution) was added to a solution of 5-chloro-2-(piperidin-4-yloxy)-pyridine in toluene at RT. Additional toluene was added and the resulting solution was stirred until crystallisation occurred. The solid formed was collected by filtration and washed with iso-hexane.

### Examples of Acids Used for this Process Include

Aqueous phosphoric acid

¹H NMR (D₂O) δ 2.10 (2H, m), 2.20 (2H, m), 3.28 (2H, m), 3.45 (2H, m), 5.15 (1H, m), 6.91 (1H, d, J 8.8 Hz), 7.78 (1H, d, 8.8 Hz), 8.10 (1H, s).
Hydrochloric acid (in propanol)—used to make the mono- and di-hydrochloride salts

Mono-HCl, ¹H NMR (d⁶-DMSO) δ 1.91 (2H, m), 2.14 (2H, m), 3.07 (2H, m), 3.21 (2H, m), 5.19 (1H, m), 6.90 (1H, d, J 9.6 Hz), 7.83 (1H, d, J 9.6 Hz), 8.21 (1H, s), 9.17 (2H, bs). M.p. 156° C.

Di-HCl, ¹H NMR (d⁶-DMSO) δ 1.93 (2H, m), 2.16 (2H, m), 3.08 (2H, m), 3.20 (2H, m), 5.20 (1H, m), 5.27 (1H, bs), 6.91 (1H, d, J 9.2 Hz), 7.83 (1H, d, J 9.2 Hz), 8.21 (1H, s), 9.35 (2H, bs). M.p. 131° C.
Trimethylacetic acid

¹H NMR (d⁶-DMSO) δ 1.10 (9H, s), 1.48 (2H, m), 1.93 (2H, m), 2.58 (2H, m), 2.95 (2H, m), 4.99 (1H, m), 6.83 (1H, d, J 8.8 Hz), 7.77 (1H, d, J 8.8 Hz), 8.18 (1H, s). M.p. 91-96° C.
Tartaric acid

¹H NMR (D₂O) δ 2.10 (2H, m), 2.21 (2H, m), 3.29 (2H, m), 3.46 (2H, m), 4.53 (2H, s), 5.17 (1H, m), 6.93 (1H, d, J 9.2 Hz), 7.80 (1H, d, J 9.2 Hz), 8.12 (1H, s).
Citric acid

Hemi-citrate, ¹H NMR (D₂O) δ 2.10 (2H, m), 2.21 (2H, m), 2.64 (1H, d, J 15.2 Hz), 2.73 (1H, d, J 15.2 Hz), 3.28 (2H, m), 3.46 (2H, m), 5.16 (1H, bs), 6.915 (1H, d, J 8.8 Hz), 7.79 (1H, d, J 8.8 Hz), 8.10 (1H, s). M.p. 88° C.
Fumaric acid

¹H NMR (CD₃OD) δ 2.05 (2H, m), 2.20 (2H, m), 3.23 (2H, m), 3.39 (2H, m), 5.30 (1H, m), 6.72 (2H, s), 6.83 (1H, d, J 8.8 Hz), 7.70 (1H, d, J 8.8 Hz), 8.11 (1H, s). M.p. 128° C.
Maleic acid

¹H NMR (d⁶-DMSO) δ 1.85 (2H, m), 2.13 (2H, m), 3.13 (2H, m), 3.27 (2H, m), 5.20 (1H, m), 6.12 (2H, s), 6.90 (1H, d, J 9.2 Hz), 7.83 (1H, d, J 9.2 Hz), 8.21 (1H, s), 8.48 (2H, bs). M.p. 96-104° C.
Benzoic acid

¹H NMR (d⁶-DMSO) δ 1.66 (2H, m), 2.02 (2H, m), 2.80 (2H, m), 3.09 (2H, m), 5.08 (1H, m), 6.86 (1H, d, J 8.8 Hz), 7.40 (2H, t, J 9.6), 7.48 (1H, d, J 9.6 Hz), 7.79 (1H, d, 8.8 Hz), 7.91 (2H, d, J 9.6 Hz), 8.19 (1H, s). M.p. 140° C.
Hydrobromic acid (aqueous)—used to make the mono- and di-hydrobromide salts Mono-HBr, ¹H NMR (d⁶-DMSO) δ 1.90 (2H, m), 2.15 (2H, m), 3.12 (2H, m), 3.25 (2H, m), 5.19 (1H, m), 6.91 (1H, d, J 8.8 Hz), 7.85 (1H, d, J 8.8 Hz), 8.21 (1H, s), 8.80 (2H, bs). M.p. 198° C.

Di-HBr, ¹H NMR (d⁶-DMSO) δ 1.91 (2H, m), 2.16 (2H, m), 3.15 (2H, m), 3.26 (2H, m), 5.21 (1H, m), 6.92 (1H, d, J 9.2 Hz), 7.84 (1H, d, J 9.2 Hz), 8.21 (1H, s), 8.76 (2H, bs). M.p. 183° C.
Carbonic acid (CO₂ gas)

Hemi-carbonate, ¹H NMR (d⁶-DMSO) δ 1.53 (2H, m), 1.94 (2H, m), 2.59-3.02 (4H), 5.04 (1H), 6.84 (1H, d, J 8.7 Hz), 7.77 (1H, d, J 8.7 Hz), 8.17 (1H, s).

### Example 2

### (RS)-5-Methyl-5-{[(phenylmethyl)thio]methyl}imidazolidine-2,4-dione

A suitable sized pressure rated reactor was charged with benzylthioacetone (95% purity) (85.26 g, 450 mmol, 1 mol eq.), water (413 mL) and 2-propanol (146 mL). The mixture was stirred for about 15 minutes to achieve homogeneity. Ammonium carbonate (49.56 g, 509 mmol, 1.13 mol eq.) and potassium cyanide (30.54 g, 460 mmol, 1.02 mol eq.) were then charged. The reaction mixture was warmed to 90° C., which induced a pressure of ca. 2.5 barg. The reaction was cooled and analysed by LC for the disappearance of starting material. After completion of the reaction, the required product was allowed to crystallize. If necessary, crystallization was induced by seeding. After crystallization, water (971.9 mL) and concentrated hydrochloric acid (96.7 g) were charged to the reaction mixture. This caused a pH change from about 11.9 to 7.4. The crystalline mass was filtered off and subsequently washed with isopropyl acetate. After drying, the title compound was obtained as a white crystalline solid in 86% yield.

¹H NMR (d⁶-DMSO) δ 10.74 (1H, s), 8.00 (1H, s), 7.35-7.20 (5H, m), 3.76 (2H, s), 2.72, 2.262 (2×1H each, Abq), 1.29 (3H, s).

### Example 3

### (S)-5-Methyl-5-{[(phenylmethyl)thio]methyl}imidazolidine-2,4-dione

(RS)-5-Methyl-5-{[(phenylmethyl)thio]methyl}imidazolidine-2,4-dione was separated into the component enantiomers using preparative chiral simulated moving bed chromatography (SMB). The same chiral stationary phase and mobile phase were used as disclosed in WO 02/074767 (page 89). The enantiomers were recovered in essentially quantitative yield.

The resulting (S)-5-benzylsulfanylmethyl-5-methyl-imidazolidine-2,4-dione (5 g) in methanolic solution was reduced in volume (to about 20 mL) under reduced pressure at 35° C. Water (40 mL) was added to the solution dropwise, maintaining the internal temperature at 35° C. After about half of the water had been added, the product started to precipitate. The mixture was allowed to cool slowly to RT and was then cooled in an ice bath to 2° C. The product (4.56 g, 91% of theoretical after SMB separation) was collected by filtration at 2° C. as a white crystalline solid. On a 5.86 kg scale, the product from this recrystallisation step was isolated in 98% yield (5.73 kg).

(S)-5-Methyl-5-{[(phenylmethyl)thio]methyl}imidazolidine-2,4-dione can also be crystallised from other methanol mixtures including methanol/toluene or methanol/dibutylether. It can be recrystallised from a range of solvents including toluene, di-isopropylether, dibutylether and water.

¹H NMR (300 MHz, d6-DMSO) δ 10.74 (1H, s), 8.00 (1H, s), 7.35-7.20 (5H, m), 3.76 (2H, s), 2.72, 2.262 (2×1H each, ABq), 1.29 (3H, s).

The chiral purity of the product was established by LC using a Hewlett Packard 1100 series HPLC fitted with a diode array detector; an Astec Chirobiotic V 50 mm×4.6 mm column; 70:30 isohexane:ethanol as mobile phase; oven temperature 55° C.; flow rate 1.0 mL/minute; detection at 210 nm; injection volume 1 μL; and run time 5 minutes. Retention times for the (S)- and (R)-isomers were 2.6 and 3.8 minutes respectively.

### Example 4

### ((S)-4-Methyl-2,5-dioxo-imidazolidin-4-yl)-methanesulfonyl chloride

Method 1

(S)-5-Benzylsulfanylmethyl-5-methyl-imidazolidine-2,4-dione (106.9 g, 427.1 mmol, 1.000 mol eq.) was dissolved in a mixture of glacial acetic acid (8 vol eq.) and water (1 vol eq.) and cooled to about 4° C. Chlorine gas (96.9 g, 3.2 mol eq.) was then passed into the well-agitated solution at a steady rate over approx. 1 h such that the reaction mixture temperature was maintained between 12 and 15° C. throughout the majority of the addition (the jacket temperature was kept at 4° C. throughout). After the reaction was complete (the mixture turns a characteristic green colour and the temperature drops sharply), the mixture was sparged with nitrogen and heated to about 30° C. to give a white slurry. The bulk of the solvent was then removed by vacuum distillation. Toluene (534.5 mL) was added and a similar volume of solvent removed by distillation under vacuum. The addition/distillation of toluene was repeated once more. Isohexane (534.5 mL) was then added to the residue and the mixture cooled to 20° C. After a stir-out, the product was collected by filtration. The collected solid was washed with isohexane (213.8 mL) and dried to constant weight in vacuo at 40° C. to give the required sulfonyl chloride as a white crystalline solid (95.5 g, 98.7%).

¹H NMR (300 MHz, d8-THF) δ, 9.91 (1H, bs), 7.57 (1H, s), 4.53, 4.44 (2×1H, each ABq), 1.52 (3H, s).

Method 2

(S)-5-Benzylsulfanylmethyl-5-methyl-imidazolidine-2,4-dione (50 g, 199.74 mmol) was dissolved in a mixture of glacial acetic acid (8 vol eq.) and water (4 mol eq.) and cooled to about 4° C. Chlorine gas was then passed into the well-agitated solution at a steady rate over approximately 1 h such that the reaction mixture temperature was maintained at approximately 12° C. throughout the majority of the addition (the Huber controller was set to maintain the reaction temp at 12° C.). After the reaction was complete (the mixture turns a characteristic green colour and the temperature drops sharply), the mixture was sparged with nitrogen and heated to about 20° C. to give a white slurry. Between half and two thirds of the solvent was then removed by vacuum distillation (at 100 mbar pressure). Iso-octane (250 mL, 5 vol eq.) was then added to the residue and the mixture cooled to 20° C. After a stir-out, the product was collected by filtration. The collected solid was washed with iso-octane (2×100 mL) and dried to constant weight in vacuo at 40-50° C. to give the required sulfonyl chloride as a white crystalline solid (41.37 g, 91%).

¹H NMR (300 MHz, d8-THF) δ, 9.91 (1H, bs), 7.57 (1H, s), 4.53, 4.44 (2×1H, each ABq), 1.52 (3H, s).

### Example 5

### (S)-5-[4-(5-Chloro-pyridin-2-yloxy)-piperidine-1-sulfonylmethyl]-5-methyl-imidazolidine-2,4-dione Process 1

### a) 5-Chloro-2-(piperidin-4-yloxy)-pyridine

5-Chloro-2-(piperidin-4-yloxy)-pyridine acetate (40 g, 0.146 mol) was slurried in iso-PrOAc (664 mL) at 30° C. To this slurry was added Na₂CO₃ (1.5 mol per litre; 196 mL, 2 mol eq.). The slurry was then rapidly stirred at 30° C. for 15 minutes. The biphasic mixture was allowed to settle, and the bottom aqueous phase was separated and discarded. The above base washing procedure was repeated twice more. The organic phase was then washed once with water (200 mL). The resulting iso-PrOAc solution was reduced in volume to approximately 300 mL by distillation under reduced pressure. The solution was then diluted with iso-PrOAc (400 mL) and again distilled down to approximately 300 mL. This procedure was repeated once more. A sample was removed for analysis of 5-chloro-2-(piperidin-4-yloxy)-pyridine content and water content. The weight or the volume of the solution was measured in order to calculate the concentration of 5-chloro-2-(piperidin-4-yloxy)-pyridine in the iPrOAc solution.

### b) (S)-5-[4-(5-Chloro-pyridin-2-yloxy)-piperidine-1-sulfonylmethyl]-5-methyl-imidazolidine-2,4-dione

Diisopropylethylamine (24.3 mL, 0.139 mol, 1 mol eq.) was added to the iso-PrOAc solution prepared in part (a) [ca. 300 mL; equivalent to 31.2 g, 0.146 mol, 1.05 mol eq. of 5-chloro-2-(piperidin-4-yloxy)-pyridine] in one portion at RT. The solution was then cooled to −15° C.

((S)-4-Methyl-2,5-dioxo-imidazolidin-4-yl)-methanesulfonyl chloride (31.65 g, 0.139 mol, 1 mol eq.) was dissolved in dry THF (285 mL) at RT with stirring. The resulting solution was then added to the iso-PrOAc solution of 5-chloro-2-(piperidin-4-yloxy)-pyridine dropwise at −15° C. over about 1.5 h. A precipitate was seen on addition of the ((S)-4-methyl-2,5-dioxo-imidazolidin-4-yl)-methanesulfonyl chloride. At the end of the addition, dry THF (32 mL) was added to the reaction mixture to wash the line and the mixture was stirred for 1 h at −15° C. It was then warmed to 20° C. over 1 h and stirred at 20° C. for 1 h further.

The reaction was quenched with 10 wt % NaHSO₄ (157 mL) with rapid stirring. After about 15 minutes, the biphasic mixture was allowed to settle, and the bottom aqueous phase was separated and discarded. This acid wash procedure was repeated once more. The organic phase was then washed with water (157 mL) using rapid stirring and allowing complete phase separation before partitioning. The reaction solution was then warmed to 40° C. and washed again with water (157 mL). THF (95 mL) was added to the organic layer that was then warmed to 40° C. and filtered at 40° C. to remove any particulate matter. The solvent volume was then reduced to about 157 mL by reduced pressure distillation with the jacket temperature at 55° C. iso-PrOAc (317 mL) was then added and the volume was again reduced to about 157 mL. Two more put-and-takes of iso-PrOAc (317 mL) were carried out. Solids began to precipitate out during the distillations and a suspension resulted. The volume was reduced to about 157 mL each time and after the final distillation a small sample of solvent was then removed from the reaction mixture for residual THF analysis. The ¹H NMR showed no THF peaks. The contents of the reaction were then cooled to 0° C. and the product was collected by filtration. The reaction vessel was washed with iso-PrOAc (63 mL) and this rinse was used to wash the product on the filter. The product was dried overnight in a vacuum oven at 40° C. The required (S)-5-[4-(5-chloro-pyridin-2-yloxy)-piperidine-1-sulfonylmethyl]-5-methyl-imidazolidine-2,4-dione was isolated as a white solid in 71% yield (41.8 g).

¹H NMR (300 MHz, d6-DMSO) δ 10.74 (1H, s), 8.20 (1H, d), 8.01 (1H, s), 7.81 (1H, dd), 6.87 (1H, d), 5.09 (1H, m), 3.52-3.35 (4H, m), 3.13 (2H, m), 2.02 (2H, m), 1.72 (2H, m), 1.33 (3H, s).

### Example 6

### (S)-5-[4-(5-Chloro-pyridin-2-yloxy)-piperidine-1-sulfonylmethyl]-5-methyl-imidazolidine-2,4-dione Process 2

### a) 5-Chloro-2-(piperidin-4-yloxy)-pyridine

5-Chloro-2-(piperidin-4-yloxy)-pyridine acetate (70 g, 257 mmol) was slurried in toluene (560 mL) at RT. 1M NaOH (420 mL) was added and the slurry was then rapidly stirred at RT for 15 min. The biphasic mixture was allowed to settle, and the bottom aqueous phase was separated and discarded. The organic phase was then washed with water (2×420 mL). A sample was removed from the organic phase and assayed for 5-chloro-2-(piperidin-4-yloxy)-pyridine.

The resulting toluene solution was then reduced in volume by distillation at reduced pressure, down to approximately 168 mL (2.4 vol eq. with respect to 5-chloro-2-(piperidin-4-yloxy)-pyridine acetate charge). The solution was then diluted with toluene (420 mL) and again distilled down to approx 168 mL (2.4 vol eq.). A sample was removed for analysis of water content.

### b) (S)-5-[4-(5-Chloro-pyridin-2-yloxy)-piperidine-1-sulfonylmethyl]-5-methyl-imidazolidine-2,4-dione

Diisopropylethylamine (38.4 mL, 220 mmol) was added to the toluene solution of 5-chloro-2-(piperidin-4-yloxy)-pyridine obtained in step (a) (containing 236 mmol) in one portion followed by dry THF (151 mL) as a line wash. ((S)-4-Methyl-2,5-dioxo-imidazolidin-4-yl)-methanesulfonyl chloride (48.7 g, 215 mmol) was dissolved in dry THF (352 mL) at RT with stirring. The resulting solution of the sulfonyl chloride was then added dropwise to the toluene/THF solution of 5-chloro-2-(piperidin-4-yloxy)-pyridine and diisopropylethylamine at RT over 1 to 2 h. A precipitate was seen on addition of the sulfonyl chloride. At the end of the addition, dry THF (50 μL) was added to the reaction mixture as a line wash. After the addition was complete, the reaction was stirred for about 30 min at RT.

The reaction was quenched with 10 wt % NaHSO₄ (251 mL) with rapid stirring for approx 15 min. The biphasic mixture was allowed to settle, when the bottom aqueous phase was separated and discarded. This acid wash procedure was repeated once more. The solvent volume was then reduced to about 220 mL by reduced pressure distillation. Toluene (300 mL) was then added and the volume was reduced to about 245 mL Solids begin to precipitate during the distillations and a suspension resulted. After the final distillation, a small sample of solvent was then removed from the reaction mixture for residual THF analysis.

The contents of the reaction mixture were then cooled to 0° C., stirred for about 30 minutes at this temperature and the product was collected by filtration. The reaction vessel was washed with toluene (100 mL) and this rinse was used to wash the product on the filter. The product was dried in a vacuum oven at 40° C. to constant weight. (S)-5-[4-(5-Chloro-pyridin-2-yloxy)-piperidine-1-sulfonylmethyl]-5-methyl-imidazolidine-2,4-dione was isolated as a white solid in typically 85 to 88% yield over the two steps.

### Example 7

### (S)-5-[4-(5-Chloro-pyridin-2-yloxy)-piperidine-1-sulfonylmethyl]-5-methyl-imidazolidine-2,4-dione Form G

A 2:1 mixture of industrial methylated spirits (IMS):water (10 vol eq.) was added to (S)-5-[4-(5-chloro-pyridin-2-yloxy)-piperidine-1-sulfonylmethyl]-5-methyl-imidazolidine-2,4-dione. The mixture was heated to reflux (about 80 to 82° C.) to obtain a solution. This solution was held at reflux for 15 minutes and then filtered. The filtrate was heated to reflux and maintained at this temperature for 15 minutes. The solution was then cooled to 20° C. at a rate of 0.5° C./minute. After stirring at 20° C. for 2 to 3 h, (S)-5-[4-(5-chloro-pyridin-2-yloxy)-piperidine-1-sulfonylmethyl]-5-methyl-imidazolidine-2,4-dione form G was collected by filtration, washed with IMS:water 2:1 (2.5 vol eq.) and dried. The product was isolated in 80 to 87% yield.

This method has been shown to reproducibly give polymorph G irrespective of the polymorphic modification of the input material. Polymorphs A, C, F and polymorphic mixtures have all been recrystallised to give form G using this method.

### Example 8

### Resolution of (RS)-5-benzylsulfanylmethyl-5-methyl-imidazolidine-2,4-dione using is (1S)-(−)-α-methyl benzylamine

A solution of NaOH (0.45 eq.) in water (4 vol eq.) was added to (1S)-α-methyl benzylamine (1.7 eq.) and (RS)-5-benzylsulfanylmethyl-5-methyl-imidazolidine-2,4-dione (50 g). The resulting suspension was heated to 83° C. The mixture was cooled slowly and seeded above RT (36° C.). Seeding is not essential but aids the ease with which the reaction mixture can be stirred and filtered. Further water (3 vol eq.) was added during the cooling cycle. The reaction was stirred at 20° C. overnight before the product was collected by filtration and washed with cyclohexane to yield (5S)-5-benzylsulfanylmethyl-5-methyl-imidazolidine-2,4-dione (1S)-α-methyl benzylamine salt in 45% yield.

LC (same conditions as for Example 3) indicated that the product had 94.5% enantiomeric purity.

¹H NMR (400 MHz, d⁶-DMSO) δ 1.23 (3H, d, J=6.7 Hz), 1.28 (3H, s), 2.61 (1H, d, J 14.1 Hz) 2.72 (1H, d, J 14.1 Hz), 3.76 (2H, s), 3.96 (1H, q, J=6.7 Hz), 7.37-7.15 (10H, m), 7.97 (1H, s).

### 2-Amino-3-benzylthio-2-methylpropionamide (VIII)

Potassium cyanide (1.07 Kg) and ammonium hydroxide solution (4.46 Kg of a 28% solution) were charged to a 20 L flask. To this mixture was added a pre-formed solution of ammonium chloride (1 Kg in 3.47 Kg water) over about 0.5 h with stirring. 1-(Benzylthio)-2-propanone (2.71 Kg) was then added. The mixture was stirred at about 40° C. for 65 h. Deionised water (1 L) was added and the phases were separated. The separated organic phase (3.13 Kg) was used directly in the next step.

To a mixture of 37% hydrochloric acid (13.3 Kg) and 48% hydrobromic acid (1.26 Kg) was added half of the material from above over about 1 h at about 5° C. The mixture was then stirred for 2 h at 5° C. and then warmed to about 30° C. and stirred overnight. The mixture was then cooled and the product collected by filtration. The filter cake was washed with ice cold conc. hydrochloric acid and acetone. The resulting material was re-suspended in acetone (10 L) and heated at reflux for a short time. The product was then collected by filtration and dried overnight under vacuum. This procedure was repeated for the second half of the starting material.

To the combined batches of material prepared above (1.66 Kg and 1.51 Kg) was added ethyl acetate (9 Kg) and a pre-formed potassium carbonate solution (9 Kg of 50 wt % in water). The mixture was stirred for 1 h and the phases were then separated. The aqueous phase was extracted with ethyl acetate (3 Kg). The combined organic phases were extracted with potassium carbonate solution (3.1 Kg of 50 wt % in water). The resulting organic phase was dried over sodium carbonate, filtered and concentrated. The resulting solid was slurried and heated at reflux in TBME for a short period. After cooling, the product was collected by filtration and dried overnight under vacuum.

2-Amino-3-benzylthio-2-methylpropionamide (VIII) (2.4 Kg, 73.2% overall) was isolated.

### Example 9

### Resolution of (RS)-2-amino-3-benzylsulfanyl-2-methyl-propionamide using (R)-(−)-mandelic acid

### (2S)-2-Amino-3-benzylsulfanyl-2-methyl-propionamide (R)-Mandelate

Methanol (20 mL, 4 vol eq.) and iso-propyl acetate (80 mL, 16 vol eq.) were mixed to give a 20% v/v solution of methanol in iso-propyl acetate.

(RS)-2-Amino-3-benzylsulfanyl-2-methyl-propionamide (5 g, 22.2 μmmol) was dissolved in the pre-mixed MeOH/ⁱPrOAc solution (13 vol eq.) and water (0.4 mL, 1 mol eq.). The resulting solution was heated to 50° C. (R)-(−)-Mandelic acid (3.39 g, 22.2 mmol, 1.0 mol eq.) was dissolved in the premixed MeOH/ⁱPrOAc solution (30 mL, 6 vol eq.) and then added to the amide solution in a controlled fashion over 1 h. During the addition the reaction temperature was maintained at 50° C. Precipitation of the product may start during the addition. The remaining premixed MeOH/ⁱPrOAc solution (about 1 vol eq.) was used as a line wash.

The suspension was then cooled slowly to 0° C. and after a stir-out period at this temperature the product was collected by filtration. The reaction vessel was returned to 20° C. and washed with ⁱPrOAc (25 mL, 5 vol eq.) for about 5 to 10 minutes. This solution was subsequently used to wash the product on the filter. The product was dried in a vacuum oven at 40° C. to constant weight. (2S)-2-Amino-3-benzylsulfanyl-2-methyl-propionamide (R)-mandelate hemihydrate was isolated as a white solid in typically 47 to 48% yield and 97 to >99% enantiomeric purity.

(2S)-2-Amino-3-benzylsulfanyl-2-methyl-propionamide (R)-mandelate hemihydrate of greater than 90% enantiomeric purity can be enantiomerically up-graded by recrystallising from iPrOAc (35 vol eq.) to yield material of >99.35% enantiomeric purity with >87% recovery.

¹H NMR (400 MHz, d⁶-DMSO) δ 1.28 (s, 3H, CH₃), 2.79 (AB q, J=140.5, 13.8 Hz, 2H SCH₂C_{q}), 3.76 (AB q, J=16.4, 13.1 Hz, 2H, ArCH₂S), 4.79 (s, 1H ArCHOH), 7.41-7.18 (m, 11H, ArH and CONH_{A}H_{B}), 7.56 (s, 1H, CONH_{A}H_{B}).

The chiral purity of the product was established by LC using a Hewlett Packard 1100 series HPLC fitted with a diode array detector; a Chiracel ChiralPak AD 25 cm×0.46 cm ID×10 μm column; solvent —0.1% v/v diethylamine in ethanol; isocratic method; oven temperature 20° C.; flow rate 1.0 mL/minute; sample diluent—purified water; detection at 210 nm; injection volume 5.0 μL; and run time 15 minutes. Retention times for the (S)- and (R)-amino amides were approximately 5.4 and 11.8 minutes respectively.

### Example 10

### Resolution of (RS)-2-amino-3-benzylsulfanyl-2-methyl-propionamide using L-tartaric acid

### (2S)-2-Amino-3-benzylsulfanyl-2-methyl-propionamide (2R,3R)-Tartrate

### Method 1

Solutions of (RS)-2-amino-3-benzylsulfanyl-2-methyl-propionamide and L-tartaric acid (0.25-1.0 eq.) in ethanol (11.75-90 vol eq.) were mixed. The resulting solid that precipitated was collected by filtration. This yielded 2-amino-3-benzylsulfanyl-2-methyl-propionamide tartrate in typically 45 to 90% yield and 50 to 92% enantiomeric purity (ep). Solvents other than ethanol (see under Method 2) could also be used.

Using L-tartaric acid (1 eq.) and EtOH (90 vol eq.), the product was isolated as a 1:1 salt in 44% yield and with 92.2% ep.

### Method 2

A mixture of (RS)-2-amino-3-benzylsulfanyl-2-methyl-propionamide and L-tartaric acid (0.25-1.0 eq.) was slurried in a minimum volume of a solvent (suitable solvents include, but are not limited to, methanol, ethanol, iso-propanol, n-butanol, iso-propyl acetate, ethyl acetate, toluene, acetonitrile and IMS). These slurries were then heated to a temperature above ambient and additional solvent was added in order to form complete solutions at various temperatures. If complete solutions were not formed in 35 vol eq. of solvent at reflux, sufficient water was added to create a solution. These solutions were then cooled to RT and the resultant solid was collected by filtration to yield 2-amino-3-benzylsulfanyl-2-methyl-propionamide tartrate in 78 to 100% yield (with respect to L-tartaric acid input and correcting for the formation of either 1:1 salts or 2:1 salts), with enantiomeric purities in the range of 50 to 60%.

These 2-amino-3-benzylsulfanyl-2-methyl-propionamide L-tartrate salts could be recrystallised from various solvents (examples of suitable solvents include, but are not limited to, (i) mixed solvent recrystallisations with either MeOH or water as a solvent and either iso-propanol, n-butanol, ethyl acetate, iso-propyl acetate, toluene, acetonitrile, acetone, THF, TBME, DCM, MIBK, diethyl ether, 2,2,4-trimethylpentane or IMS as antisolvents; or (ii) direct recrystallisation from ethanol, methanol, IMS or water).

Recrystallisation typically gave yields in the range 1 to 99% with enantiomeric purities in the range 50 to 96%.

Recrystallisation from MeOH/MIBK (20/29.5 vol eq. respectively) gave (2S)-2-amino-3-benzylsulfanyl-2-methyl-propionamide (2R,3R)-tartrate in 75% yield and with an enantiomeric purity of 96.63% (the starting material was ca. 50% ep).

Chiral purity was established by LC, as for Example 9.

¹H NMR (300 MHz, d⁶-DMSO) δ 1.35 (3H, s, CH₃), 2.69 (1H, d, J 13.7 Hz, S—CH₂), 3.00 (1H, d, J 13.7 Hz, S—CH₂), 3.79 (2H, s, S—CH₂), 3.98 (2H, s, CHOH), 7.22-7.36 (5H, m, Ar-H), 7.44 (1H, s, NH), 7.63 (1H, s, NH).

### Example 11

### Biocatalytic Resolutions

### 1. Using Rhodococcus erthoplis Amidase

(RS)-2-Amino-3-benzylsulfanyl-2-methyl-propionamide hydrochloride salt (5 g, 0.019 mol) was dissolved in 0.1N pH 8.00 borate buffer (100 mL) to give a solution with pH about 4.0. The pH was adjusted to 7.00 with 2N KOH solution and Rhodococcus erthoplis amidase was added as a cross linked enzyme aggregate (CLEA) (1 mL of a suspension in buffer, 25 units/mL specific activity). The reaction mixture was stirred at 35° C. for 1.5 h. HPLC showed 25 to 30% conversion of the amide to the corresponding amino acid. The pH was adjusted to 1.00 with concentrated hydrochloric acid and then filtered to remove the enzyme CLEA. The pH was adjusted to 11 with 2N KOH solution and extracted with DCM (1×100 mL; 1×40 mL). The combined DCM extracts were dried and evaporated to give (R)-2-amino-3-benzylsulfanyl-2-methyl-propionamide as a white solid (2.67 g) in 62% yield. The enantiomeric purity was found to be 67% (R)-ent by chiral HPLC (34% ee).

The aqueous layer was treated with concentrated hydrochloric acid to pH 6.80 and maintained at pH 6.80 for 1 h by the occasional addition of 2N KOH solution. The resulting white crystals were collected by filtration, washed with pH 7.00 buffer and dried at 40° C. overnight to give (S)-2-amino-3-benzylsulfanyl-2-methyl-propionic acid (1.2 g, 27% yield). The enantiomeric purity was found to be 99% (S)-ent (98% ee).

### 2. Using Pseudomonas fluorescens AL45 Amidase (WO 2005/123932)

a) Growth of the Microorganism

Ps fluorescens AL45 was grown in a 10 L fermenter in 5 L of mineral salts medium pH 7.2 supplemented with yeast extract (2 g/L) and lactamide (2.5 g/L) and maintained at 28° C. The fermenter was aerated at 5 L/min and stirred at 400 rpm for 24 h. Cells were harvested by centrifugation and the recovered cells were washed by re-suspension in 100 mM phosphate buffer pH 7.2 and re-centrifuged. The recovered cells were stored at 4° C. overnight prior to use in the biotransformation.

b) Biotransformation Conditions

The cell pellet was re-suspended in phosphate buffer (100 mM, pH 7.2, 1 L) and to this was added (RS)-2-amino-3-benzylsulfanyl-2-methyl-propionamide (5 g). The mixture was stirred and incubated at 28° C. and samples were removed periodically for analysis. After 8.5 h, quantitative HPLC indicated that the reaction had reached 50% hydrolysis. Chiral analysis (LC) indicated (R)-2-amino-3-benzylsulfanyl-2-methyl-propionamide 96% ee; and (S)-2-amino-3-benzylsulfanyl-2-methyl-propionic acid 97.5% ee.

c) Reaction Work-Up

Cells were removed from the biotransformation broth by centrifugation and the supernatant (approximately 1000 mL) adjusted to pH 10.5 using 20% sodium carbonate solution (final volume approximately 1200 mL). The alkaline supernatant was extracted with TBME (750 mL), any emulsion being retained with the organic phase. The TBME phase was mixed with acetone (approximately 250 mL) which resulted in the formation of a precipitate. Meanwhile the aqueous phase was re-extracted with TBME (750 mL) and again the organic phase was mixed with acetone (approximately 250 mL). This resulted in the separation of a small aqueous layer which was combined with the rest of the aqueous phase. The two TBME/acetone phases were combined, dried (Na₂SO₄), filtered and the solvent was removed to give (R)-2-amino-3-benzylsulfanyl-2-methyl-propionamide as an off-white crystalline solid (1.7 g) in 68% yield. The enantiomeric purity was >99% (R)-ent.

The aqueous phase was adjusted to pH 6.8 using 2M HCl and concentrated by freeze drying to approximately 300 mL. Upon thawing a white slurry was produced. The crystalline solid was recovered by filtration and dried overnight at 37° C. to give (S)-2-amino-3-benzylsulfanyl-2-methyl-propionic acid as fine white crystals (1.8 g) in 72% yield, 98% purity by HPLC. The enantiomeric purity was >99% (S)-ent.

### 3. Using Mycobacterium neoarum L-amidase

Using an analogous procedure to that described for Rhodococcus erthoplis, (RS)-2-amino-3-benzylsulfanyl-2-methyl-propionamide can be resolved using an L-specific amidase from Mycobacterium neoarum. This method is complementary to those described in (1) and (2) above in that (R)-2-amino-3-benzylsulfanyl-2-methyl-propionic acid and (S)-2-amino-3-benzylsulfanyl-2-methyl-propionamide are produced in ˜99% ee.

### 4. (S)-5-Benzylsulfanylmethyl-5-methyl-imidazolidine-2,4-dione via resolution of (RS)-3-benzylsulfanyl-2-methyl-2-ureido-propionic acid

### (RS)-3-Benzylsulfanyl-2-methyl-2-ureido-propionic acid

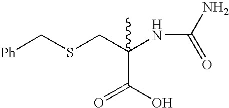

(RS)-2-Amino-3-benzylsulfanyl-2-methyl-propionic acid (350 g) was suspended in water (2.6 L) and THF (2.6 L), potassium cyanate (504 g) was added and the reaction was stirred at 25° C. overnight. Additional potassium cyanate (75 g) was then added and the reaction was stirred for a further 6 h. Stirring was then stopped and the reaction was cooled at 0° C. overnight. The resulting slurry was filtered and the filtrate was concentrated under reduced pressure. The concentrate was acidified to pH 2 with 6% aqueous HCl solution with rapid stirring. After 1 h the resulting solid was collected by filtration and was then sequentially re-slurried in water (4 L) and methanol (4 L). After drying under vacuum, the product was purified using silica chromatography. Impurities were eluted using DCM/THF while the product was eluted using THF/AcOH followed by MeOH. Removal of the solvent followed by trituration with TBME gave 3-benzylsulfanyl-2-methyl-2-ureido-propionic acid (151 g) as an off-white solid.

(RS)-3-Benzylsulfanyl-2-methyl-2-ureido-propionic acid (9 g, about 90% pure, equivalent to 30 mmol) was added to sodium sulphite solution (5 mmol, 100 mL). KOH (2.5 g, 44 mmol) was added. The solution was stirred until the acid had dissolved and was then filtered. The pH was adjusted from 12.5 to 7.00 with glacial acetic acid. MnCl₂.4H₂O solution was added (10 mmolar, 10 mL). The solution was heated to 40° C. under N₂ and D-specific hydantoinase added (1 mL suspension in buffer, Roche Hydantoinase 2 recombinant in E. coli., 300 units per mg, total protein 70 mg). The reaction mixture was stirred at 40° C. under N₂ During the reaction the pH was maintained at 7.0 by titration of 5% v/v acetic acid solution.

| Time | pH | Titrant added | Conversion by HPLC |
|---|---|---|---|
| 1.0 h | 7.00 | 10 mL | 32% |
| 14 h | 7.00 | 17 mL | 44% |

During the reaction a thick white precipitate formed. The mixture was cooled to ambient temperature and stirred for 3 h, then filtered and washed with 0.1N pH 7 potassium phosphate buffer. The solid was dried overnight at 40° C. The product was isolated as a white powder (3.3 g) in 42% yield, 100% chemical purity by HPLC. Chiral HPLC analysis showed the product to be 100% (S)-ent.

The chiral purity was established using a 25 cm×4.6 mm Chiralpack AD column, 30° C., eluent MeOH+0.1% v/v HCO₂H, flow 1 mL min⁻¹, detection UV at 220 nM. Rt(S)-enantiomer 4.42 mins; Rt (R)-enantiomer 9.21 mins.

### 5. Resolution of (RS)-3-Benzylsulfanyl-2-methyl-2-(2,2,2-trifluoro-acetylamino)-propionic acid using L-Acylase from Aspergillus sp.

### (RS)-3-Benzylsulfanyl-2-methyl-2-(2,2,2-trifluoro-acetylamino)-propionic acid

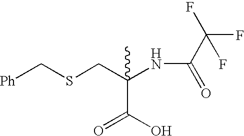

(RS)-2-Amino-3-benzylsulfanyl-2-methyl-propionic acid (5 g, 0.022 mol) was slurried in trifluoroacetic anhydride (15 mL) and DCM (15 mL). The slurry was cooled to −20° C. and triethylamine (2.5 g, 0.025 mole) added dropwise. The reaction was allowed to warm to RT. HPLC showed complete conversion to the amide. The mixture was diluted with water (30 mL) and DCM (30 mL). The aqueous layer was discarded and the organic layer washed with water (3×50 mL). The organic layer was separated and evaporated to leave an oil. This material was dissolved in acetonitrile and the solvent evaporated in vacuo.

Yield 7.2 g pale yellow oil. (˜100% yield).

MS M⁺, 321 (100%).

¹H NMR (CDCl₃) δ 7.4 (1H, brs), 7.30 (5H, m), 3.85 (2H, m), 3.4 (1H, d), 3.05 (1H, d), 1.75 (3H, s).

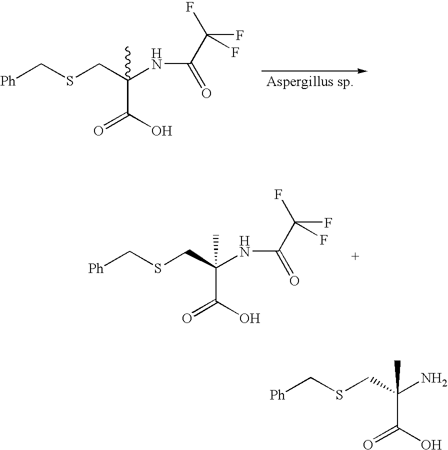

CoCl₂.6H₂O (0.25 g, 0.001 mol) was dissolved in borate buffer (100 mL, pH 8.0, 0.1N). The solution was degassed and the (RS)-trifluoroacetamide (7.00 g) was added as an oil. The mixture was stirred under N₂ at 45° C. adding saturated Na₂CO₃ solution until the pH reached 8.00 (ca. 35 mL added). L-amino acid acylase (0.7 gram, L-Acylase from Aspergillus sp., 30,000 units g⁻¹) was dissolved in pH 8.00 buffer (5 mL) and added to the reaction. The reaction was stirred for 30 h at 45° C. under N₂, maintaining the pH ˜7.50. The pH was adjusted to 7.00 with HOAc and a precipitated solid was collected by filtration, washed with pH 7.00 buffer and dried in vacuo. The (R)-amino acid (1.7 g, 34%) was isolated as an off-white solid. Chiral HPLC showed the material to be 98% (R)-enantiomer (96% ee). Some uncrystallised amino acid remained in the filtrate.

The filtrate was adjusted to pH 1 with conc. HCl and extracted with DCM (2×75 mL). HPLC showed the DCM extracts to contain no (R)-amino acid.

The DCM was dried (Na₂SO₄) and evaporated to leave (S)-3-benzylsulfanyl-2-methyl-2-(2,2,2-trifluoro-acetylamino)-propionic acid (2.5 g, 35%) as a waxy off-white solid.

This material was hydrolysed to the (S)-amino acid using MeOH/water/KOH in 98% yield. The product (S)-amino acid was shown by HPLC to be 96% ee.

### Example 12

### Biocatalytic Resolutions using Desymmetrisation

### 2-Benzylsulfanylmethyl-2-methyl-malonic acid diethyl ester

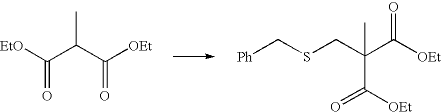

To a cooled solution of diethyl methylmalonate (79.9 g) and benzylthiobromomethane (110.4 g) in 2-methyl-THF (480 mL) was added potassium butoxide (53.6 g) portion-wise over about 2 h while maintaining the temperature below 0° C. The mixture was then allowed to warm to RT and stirred overnight. The reaction was diluted with water (320 mL) and the phases were separated. The organic phase was dried (Na₂SO₄), filtered and the solvent was removed under vacuum. The resulting oil was purified using column chromatography (eluent DCM/hexane) giving the title compound as a yellow oil (109 g, 78%).

¹H NMR (CDCl₃) δ 1.23 (6H, t), 1.47 (3H, s), 2.98 (2H, s), 3.72 (2H, s), 4.17 (4H, q), 7.2-7.3 (5H, m).

### 2-Benzylsulfanylmethyl-2-methyl-malonamide

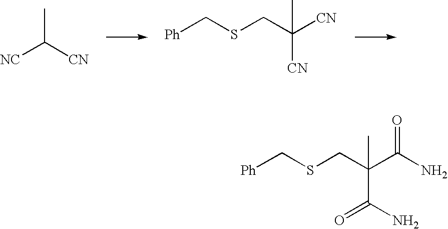

A mixture of methylmalononitrile (6.6 g) and tetrabutylanmonium bromide (1.06 g) in DCM (50 mL) was cooled to about 0° C. To this mixture was added potassium t-butoxide (9.2 g) followed by the slow addition of benzylthiobromomethane (17.89 g). The reaction was allowed to warm to RT overnight when it was diluted with brine (100 mL). The phases were separated and the aqueous phase was extracted a further three times with DCM (3×25 mL). The combined organic phase was dried (MgSO₄), filtered and concentrated under vacuum. The resulting brown oil was purified using column chromatography (eluent ethyl acetate/hexane). 2-Benzylsulfanylmethyl-2-methyl-malononitrile was isolated as a colourless solid (9.76 g, 52%).

¹H NMR (CDCl₃) δ 1.82 (3H, s), 2.89 (2H, s), 4.00 (2H, s), 7.26-7.37 (5H, m).

2-Benzylsulfanyl-2-methyl malononitrile (2.3 g) was dissolved in t-butanol (30 mL). The solution was warmed to 60° C. when powdered potassium hydroxide (10 g) was added portion-wise. After heating at 60° C. overnight, the reaction was diluted with brine and extracted with DCM (3×25 mL). The combined organic phase was dried (MgSO₄), filtered and concentrated under vacuum to give the title compound as a off-white solid.

¹H NMR (d⁶-DMSO) δ 1.38 (3H, s), 2.98 (2H, s), 3.78 (2H, s), 6.94 (4H, s), 7.15-7.43 (5H, m).

### Alternative Preparation Using Nitrile Hydratase

(RS)-2-Benzylsulfanyl-2-methyl malononitrile (4 g, 0.19 mol) was dissolved in DSMO (15 mL). Freeze-dried cells (0.75 g) containing nitrile hydratase (Zyanotase™) were slurried in 0.1N pH 7 phosphate buffer (120 mL) and the DMSO solution of the dinitrile added. The reaction was shaken at 38° C. for 2 days and then filtered. The crystallised product was dissolved in acetonitrile (70 mL) and filtered to remove cells. The solvent was removed by evaporation and the crystallised product washed with water and dried to give 2-benzylsulfanylmethyl-2-methyl-malonamide (3 g, 65%). Extraction of the aqueous filtrate from the reaction with DCM (100 mL) followed by evaporation yielded a further crop of product (750 mg, 16%).

### (S)-2-Amino-3-benzylsulfanyl-2-methyl-propionic acid via desymmetrisation

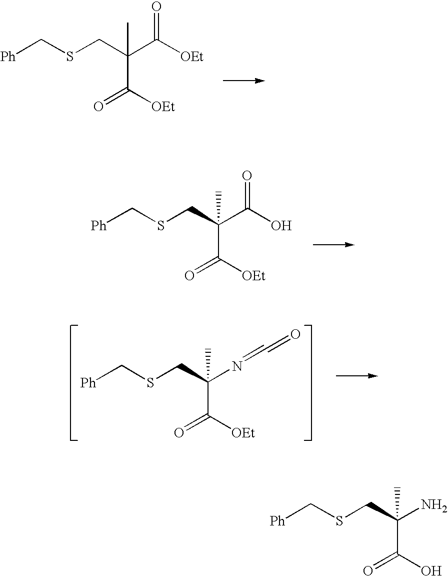

2-Benzylsulfanylmethyl-2-methyl-malonic acid diethyl ester (5 g) was slurried in 0.1M pH 7 phosphate buffer and Bacillus licheniformis protease (1 g) was added and the reaction mixture was stirred at 35° C. The pH was maintained by the addition of 2M ammonia via pH stat. After 4 days, the reaction was extracted with ethyl acetate (2×50 mL) to remove unreacted diester, acidified to pH 4 with dilute HCl and extracted with 1,2-dichloroethane (2×50 mL). The dichloroethane phase containing the product was dried (Na₂SO₄) and filtered. To the dichloroethane phase, was added triethylamine (1.6 g) followed by diphenylphosphoryl azide (PhO)₂P(O)N₃ (4.4 g). The reaction was heated at reflux overnight, mixed with 5M HCl (50 mL) and heated at reflux for a further 6 h. Chiral HPLC analysis of the acidic aqueous phase, into which the product amino acid was extracted, showed that the (S)-amino acid was present and the ee was 100%. That is, no (R)-amino acid could be detected.

The chiral purity was established using a 25 cm×4.6 mm Chirobiotic T column, 30° C., eluent 20% v/v water in EtOH, flow 1 mL min⁻¹, detection UV at 220 nM. Rt (R)-enantiomer 6.86 mins; Rt (S)-enantiomer 8.21 mins.

### (S)-5-Benzylsulfanylmethyl-5-methyl-imidazolidine-2,4-dione via Desymmetrisation

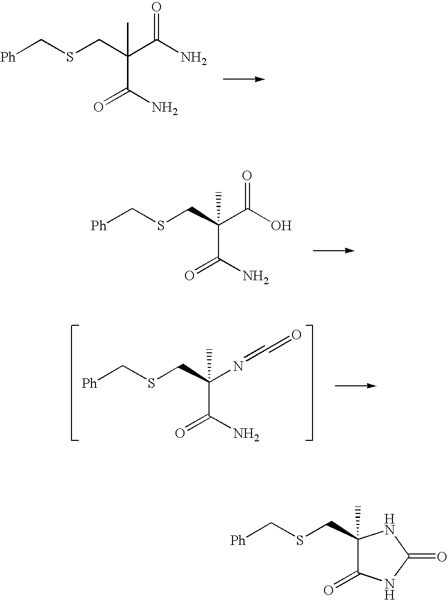

2-Benzylsulfanylmethyl-2-methyl-malonamide (2 g) was dissolved in DMSO (10 mL) at 50° C. The resulting solution was added to 0.1M pH 7 phosphate buffer (100 mL). To this was added Rhodocuccus erthoplis amidase (2 mL of a 25 units/mL suspension of cross linked enzyme aggregates (CLEA)). After stirring at 35° C. for 4 days, the CLEA was removed by filtration, the filtrate was acidified to pH 4 with conc. HCl and the product acid/amide was extracted with iso-propyl acetate (3×50 mL). Chiral HPLC showed that the mono acid had an ee of 93%. The solvent was removed under vacuum and replaced with 1,2-dichloroethane (50 mL). To this solution was added triethylamine (0.79 g) followed by diphenylphosphorylazide (2.2 g). The reaction was heated at reflux for 18 h, cooled, washed with dilute HCl (0.1M, 2×25 mL) and concentrated under vacuum. The crude product was shown to be the (S)-enantiomer and 93% ee by chiral HPLC. The product hydantoin was purified using column chromatography (eluent ethyl acetate) and was recrystallised from ethanol to give the title compound (1 g, 49%) as a colourless solid. The ee had been up-graded to 97%.

The chiral purity was established using a 25 cm×4.6 mm Chiralpack AD column, 30° C., eluent MeOH+0.1% v/v HCO₂H, flow 1 mL min⁻¹, detection UV at 220 nM. Rt (S)-enantiomer 4.42 mins; Rt (R)-enantiomer 9.21 mins.

### Example 13

### a) (R)-2-Benzylsulfanylmethyl-2-methyl-4-phenyl-oxazolidine

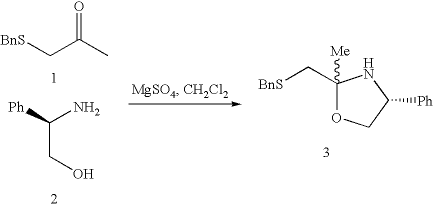

4 Å Molecular sieves (3.6 g) and benzylthioacetone (10.0 mmol, 1.65 mL, 1.80 g) were added to a solution of (R)-(−)-phenylglycinol (1.00 eq, 10.0 mmol, 1.37 g) in toluene (170 mmol, 18.0 mL, 15.7 g) at ambient temperature. The mixture was heated to 50° C. and stirred at this temperature for 24 h. Following filtration through a 50 mm no. 3 sinter funnel, a solution of the required oxazolidine was obtained. This solution was used in subsequent experiments without further purification. For analytical purposes, a small sample of this solution was evaporated to dryness to give a light coloured oil. This material was analysed by ¹H NMR spectroscopy (CDCl₃, 400 MHz) showing that the oxazolidine existed as a 54:46 mixture of (2R,4R)- and (2S,4R)-isomers.

¹H NMR (CDCl₃, 400 MHz):

(2R,4R)-isomer: δ 1.43 (3H, s); 2.83 (2H, AB, J 14.5 Hz, Δ 119.5 Hz); 3.65 (1H, t, J 7.5 Hz); 3.86 (2H, AB, J 13.1 Hz, Δ 40.7 Hz); 4.24 (1H, t, J 7.5 Hz); 4.51 (1H, brt, J 7.5 Hz); 7.2-7.5 (10H, m).

(2S,4R)-isomer: δ 1.50 (3H, s); 2.70 (2H, AB, J 13.7 Hz, Δ 43.1 Hz); 3.65 (1H, t, J 7.5 Hz); 3.86 (2H, AB, J 12.8 Hz, Δ 11.3 Hz); 4.24 (1H, t, J 7.5 Hz); 4.46 (1H, brt, J 7.5 Hz); 7.2-7.5 (10H, m).

### b) (R)-3-Benzylsulfanyl-2-methyl-2-((R)-1-phenyl-2-trimethylsilanyloxy-ethylamino)-propionitrile

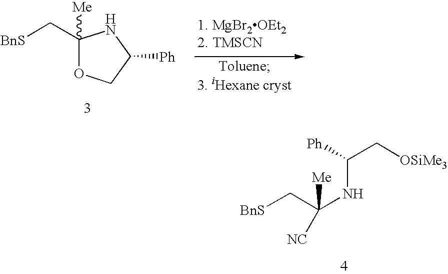

To a solution of (R)-2-Benzylsulfanylmethyl-2-methyl-4-phenyl-oxazolidine (1.00 eq, 100 mmol, 29.9 g) in toluene (4.25 mol, 449 mL, 391 g) at −20° C. was added magnesium bromide diethyl etherate (99 wt/wt %, 5.00 mmol, 1.30 g) followed by trimethylsilyl cyanide (105 mmol, 14.1 mL, 10.4 g). The mixture was stirred at −20° C. for 18 h and was then allowed to warm to 0° C. and was washed with water (60 mL, 2 vol eq.). The solvent was removed using a rotary evaporator (bath temperature 44° C.) to a volume of 60 mL (2 vol eq.) and was then diluted with iso-hexane (240 mL, 8 vol eq.). The mixture was heated to reflux to give a clear yellow solution and then allowed to cool with stirring overnight.

The solid product was collected by filtration, washed with iso-hexane (30 mL, 1 vol eq.) and dried to give the title compound (21.5 g, 54.0 mmol, 54%).

¹H NMR (CDCl₃, 400 MHz): δ 0.16 (9H, s); 0.98 (3H, s); 2.69 (2H, AB, J 14.2 Hz, Δ 43.1 Hz); 3.33 (1H, brs); 3.42 (1H, dd, J 10.6, 9.8 Hz); 3.66 (1H, dd, J 10.6, 4.2 Hz); 3.99 (2H, AB, J 13.5 Hz, Δ 32.4 Hz); 4.03 (1H, dd, J 9.8, 4.2 Hz); 7.0-7.5 (10H, m).

### c) (3R,5R)-3-Benzylsulfanylmethyl-3-methyl-5-phenyl-morpholin-2-one

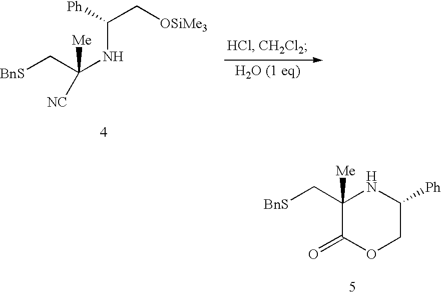

Hydrogen chloride gas was bubbled for 15 minutes through a solution of (R)-3-benzylsulfanyl-2-methyl-2-((R)-1-phenyl-2-trimethylsilanyloxy-ethylamino)-propionitrile (1.00 eq., 10.0 mmol, 3.99 g) in DCM (622 mmol, 39.9 mL, 52.8 g) held at −20° C. Water (10.0 mmol, 180 μL, 180 mg) was added and hydrogen chloride was bubbled through for a further 20 minutes. The mixture was allowed to warm to ambient temperature then stirred at ambient temperature for 12 h before being evaporated to dryness under reduced pressure. The mixture was dispersed in DCM (50 mL) and stirred with saturated aqueous sodium hydrogen carbonate (50 mL) for 30 minutes. The organic layer was separated, dried (Na₂SO₄) and evaporated to dryness under reduced pressure to give the title compound as a light brown crystalline solid.

¹H NMR (d₆-DMSO, 400 MHz); δ 1.66 (s, 3H); 2.80 (AB, J 13.1 Hz, Δ 71.1 Hz, 2H); 3.02 (brs, 1H); 3.91 (s, 2H); 4.15 (t, J 10.4 Hz, 1H); 4.30 (1H, dd, J 10.3, 2.8 Hz); 4.40 (dd, J 10.8, 2.8 Hz, 1H); 7.2-7.4 (m, 5H).

¹³C NMR (d₆-DMSO, 100 MHz); δ 25.4; 37.1; 42.7; 51.8; 62.6; 74.8; 126.7; 127.3; 128.0; 128.4; 128.4; 128.8; 138.5; 138.9; 172.0).

IR (neat solid) 3312, 3061, 3030, 2978, 2921, 2322 (w), 1730 (s), 1494, 1453, 1405, 1369, 1325, 1289, 1276, 1237, 1202 (m), 1167 (s), 1071, 1053 (m), 1029, 758, 733, 697 (s) cm⁻¹

MS (CI), m/z (%); 350 (M+Na, 30), 328 (M+H, 100).

### d) (R)-5-Benzylsulfanylmethyl-1-((R)-2-hydroxy-1-phenyl-ethyl)-5-methyl-imidazolidine-2,4-dione and Carbamic acid (R)-2-((R)-5-benzylsulfanylmethyl-5-methyl-2,4-dioxo-imidazolidin-1-yl)-2-phenyl-ethyl ester

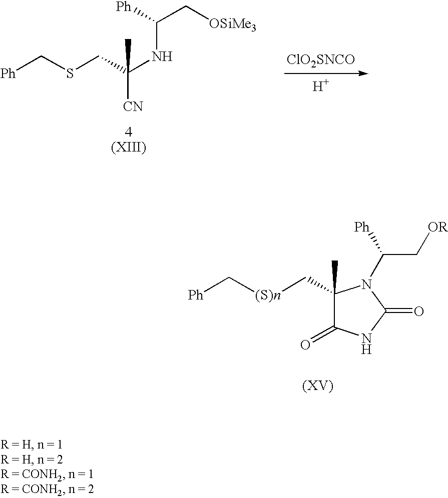

Chlorosulfonyl isocyanate (2.26 mmol, 197 μL, 320 mg) was added to a solution of (R)-3-benzylsulfanyl-2-methyl-2-((R)-1-phenyl-2-trimethylsilanyloxy-ethylamino)-propionitrile (2.26 mmol, 900 mg) in DCM (9.00 mL) at −55° C. The mixture was stirred at −55 to −40° C. for 2 h, then allowed to warm to ambient temperature and the solvent was removed by evaporation under reduced pressure to give an orange foam. This material was heated at reflux in 1M hydrochloric acid (9.0 mL) for 2 h, then allowed to cool to ambient temperature and extracted with DCM (10 mL). The DCM extract was dried (Na₂SO₄) and evaporated to dryness under reduced pressure to give an orange-brown foam. Purification by chromatography on biotage 40M column (eluent gradient 90:10 iso-hexane:ethyl acetate to ethyl acetate) gave a colourless foam which hardened on high vacuum drying to a glassy solid. Crystallisation from toluene (1.8 mL) gave a mixture of the hydantoin (XV; R=H, n=1) and the disulphide analogue (XV; R=H, n=2), along with the corresponding urethanes (XV; R=CONH₂, n=1) and (XV; R=CONH₂, n=2) in a ratio of 60:25:13:2 as a white solid (150 mg, approx. 0.41 mmol, 18%).

Hydantoin (XV; R=H, n=1)

¹H NMR (CDCl₃, 300 MHz); δ 1.52 (3H, s); 2.50 (2H, AB, J 14.4 Hz, Δ 51.0 Hz); 3.02 (1H, t, J 6.0 Hz); 3.41 (2H, AB, J 13.3 Hz, Δ 44.4 Hz); 3.89 (1H, ddd, J 11.5, 6.0, 4.5 Hz); 4.26 (1H, dd, J 9.0, 4.5 Hz); 4.55 (1H, ddd, J 11.5, 9.0, 6.0 Hz); 7.1-7.5 (10H, m); 8.50 (1H, brs).

¹³C NMR (CDCl₃, 75 MHz); δ 21.7; 36.5; 37.3; 60.6; 63.4; 68.8; 127 to 140 (12 peaks); 156.9; 175.9.

IR 3189 (br); 3061 (m); 1765 (m); 720 (s); 1495 (w); 1432; 1374 (m); 1265; 1242; 1199; 1130; 1070; 1045; 851; 764 (w); 699 (m) cm⁻¹.

MS (CI), m/z(%); 371 (M+H, 70); 251 (M-PhCH₂CH₂OH, 100).

### e) (R)-5-Benzylsulfanylmethyl-1-((R)-2-hydroxy-1-phenyl-ethyl)-5-methyl-imidazolidine-2,4-dione

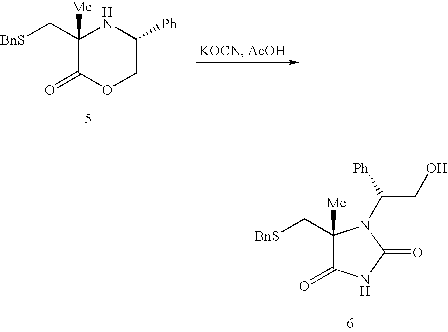

(3R,5R)-3-Benzylsulfanylmethyl-3-methyl-5-phenyl-morpholin-2-one (1.00 eq., 2.69 mmol, 880 mg) was dissolved in acetic acid (9 mL) and charged to a stainless steel ‘bomb’ type vessel. Potassium cyanate (26.9 mmol, 2.18 g) was charged, the vessel sealed immediately and the contents stirred at ambient temperature for 4 days. The mixture was poured into water (18 mL) and extracted twice with DCM (18 mL). The organic extracts were dried (Na₂SO₄) and evaporated to dryness under reduced pressure to give a brown oil. Analysis of this material by LC-MS showed a mixture of (R)-5-benzylsulfanylmethyl-1-((R)-2-hydroxy-1-phenyl-ethyl)-5-methyl-imidazolidine-2,4-dione and (3R,5R)-3-benzylsulfanylmethyl-3-methyl-5-phenyl-morpholin-2-one in an approximate ratio of 40:60.

### f) (R)-5-Benzylsulfanylmethyl-5-methyl-imidazolidine-2,4-dione

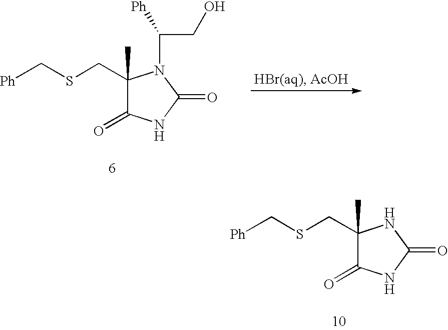

(R)-5-Benzylsulfanylmethyl-1-((R)-2-hydroxy-1-phenyl-ethyl)-5-methyl-imidazolidine-2,4-dione (1.00 eq., 1.00 mmol, 370 mg) was dissolved in acetic acid (140 mmol, 8.00 mL, 8.38 g). 48% Hydrobromic acid (133 mmol, 15.0 mL, 22.5 g) was added and the mixture heated at reflux for 4 h. The mixture was cooled to 0° C., ice (100 g) was added, the mixture was neutralised to pH 7 by addition of 0.880 SG aqueous ammonia and extracted with DCM (100 mL). The organic extract was dried (Na₂SO₄) and evaporated to dryness under reduced pressure to give the title compound as a brown gum (100 mg, 0.624 mmol, 62%).

### Example 14

### a) tert-Butyl N-benzylidenealaninate

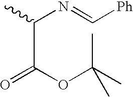

t-Butyl (DL)-alaninate hydrochloride (5 g, 27 mmol) was slurried in DCM (100 mL) and magnesium sulphate (6.50 g, 51 mmol), benzaldehyde (2.86 g, 27 mmol) and triethylamine (2.73 g, 27 mmol) were added. This mixture was stirred for 16 h at RT under a nitrogen atmosphere. Magnesium sulphate (0.81 g, 0.67 mmol) was added to the mixture and the reaction was stirred for another 24 h. The mixture was filtered and the filtrate washed with water (2×50 mL). The organic layer was dried (MgSO₄) and evaporated to give the title compound as a colourless oil (5.54 g, 83%).

¹H-NMR (300 MHz, CDCl₃) δ 1.47 (9H, s), 1.50 (3H, s), 4.01-4.08 (1H, q), 7.39-7.43 (3H, m), 7.76-7.79 (2H, m), 8.31 (1H, s).

### b) tert-butyl S-benzyl-N-benzylidene-2-methylcysteinate

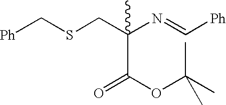

Sodium hydride (0.17 g, 4.3 mmol) was added to a solution of tert-butyl N-benzylidenealaninate (0.5 g, 2 mmol) in THF (5 mL) at RT. The mixture was stirred for 30 mins and then bromomethylsulfanylmethyl-benzene (0.52 g, 2.4 mmol) was added. The mixture was stirred at RT for 48 h and then quenched with water (10 mL). The aqueous layer was extracted with ethyl acetate (2×50 mL). The ethyl acetate layer was washed with water (10 mL), dried (MgSO₄) and evaporated to dryness to give the title compound as a yellow oil (0.60 g, 86%).

Chiral HPLC analysis showed the presence of a 1:1 mixture of enantiomers:

| Column | Chiral Pak AD | |
|---|---|---|
| Mobile phase | 1% i-PrOH in isohexane | |
| Oven temp | 15° | C. |
| Flow rate | 0.5 | mL/min |
| Detection | 210 | nm |
| Run time | 30 | min |

¹H-NMR (300 MHz, CDCl₃) δ 1.47 (9H, s), 1.48 (3H, s), 2.89-3.05 (2H, m), 3.78 (2H, s), 7.2-7.50 (5H, m), 7.39-7.43 (3H, m), 7.76-7.79 (2H, m), 8.31 (1H, s).

### c) tert-Butyl S-benzyl-N-benzylidene-2-methyl-D(R)-cysteinate

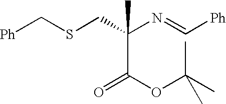

Powdered potassium hydroxide (5.90 g, 105 mmol) and chiral phase transfer catalyst (−)-O-allyl-N-(9-anthracenylmethyl)cinchonidinium bromide (1.27 g, 2.1 mmol) were added to tert-butyl N-benzylidenealaninate (5 g, 21 mmol) in toluene (50 mL) at −20° C. The mixture was stirred at this temperature for 1 h. The reaction mixture was then cooled to −30° C. and a solution of bromomethylsulfanylmethyl-benzene (11.6 g, 53 mmol) in toluene (50 mL) was added dropwise over 1 h. The mixture was stirred at this temperature for 1 h and then allowed to come to RT. The reaction was filtered through celite and washed with toluene (100 mL). The solvent was evaporated to give a crude residue (19 g) that was taken up in toluene (100 mL) and washed with water (2×25 mL). The organic layer was evaporated to give the title compound. The enantiomeric purity at this stage was not determined. This material was used directly in the next step.

¹H-NMR (300 MHz, CDCl₃) δ 1.47 (9H, s), 1.48 (3H, s), 2.89-3.05 (2H, m), 3.78 (2H, s), 7.2-7.50 (5H, m), 7.39-7.43 (3H, m), 7.76-7.79 (2H, m), 8.31 (1H, s).

### d) S-Benzyl-2-methyl-D(R)-cysteine hydrochloride

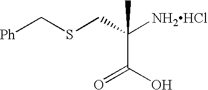

THF (50 mL) and 6M hydrochloric acid (50 mL) were added to the crude product from step (c). The mixture was stirred at RT for 1 h. The organic solvent was evaporated off and the remaining aqueous layer was washed with ethyl acetate (3×50 mL). The combined organic layers were washed with 6M hydrochloric acid (50 mL). The combined aqueous layers were heated at 60° C. for 24 h. The solvent was evaporated under reduced pressure and the resulting material was slurried in DCM (100 mL) and the solid was collected by filtration. This solid was washed with DCM (100 mL) to yield the title compound as a solid (3.6 g, 64% yield over two steps). Chiral HPLC analysis showed the product to have 96.90% enantiomeric purity.

LC Method:

| Column | Astek Chirobiotic | |
|---|---|---|
| Mobile phase | 50:50% ethanol/water | |
| Oven temp | 15° | C. |
| Flow rate | 1 | mL/min |
| Detection | 254 | nm |
| Run time | 10 | min |

¹H-NMR (400 MHz, dmso-d₆) δ 1.44 (3H, s), 2.87 (1H, d), 2.98 (1H, d), 3.8-3.85 (2H, m), 7.3-7.39 (5H, m), 8.50 (3H, s).

MS m/z, 226 [MH]⁺.

### Example 15

### a) tert-Butyl S-benzyl-N-benzylidene-2-methyl-L(S)-cysteinate

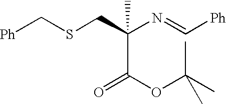

Powdered potassium hydroxide (1.17 g, 21 mmol) and chiral phase transfer catalyst (+)-O-allyl-N-(9-anthracenylmethyl)cinchonidinium bromide (0.26 g, 0.42 mmol) were added to tert-butyl N-benzylidenealaninate (1 g, 4.2 mmol) in toluene (15 mL) at −30° C. A solution of bromomethylsulfanylmethyl-benzene (4.65 g, 21 mmol) in toluene (5 mL) was added to this mixture at −30° C. The reaction was stirred at −15° C. for 16 h. The mixture was filtered through celite and washed with toluene (40 mL). The filtrate was washed with water (5 mL) and the organic layer was evaporated to dryness to give a dark brown oil. The enantiomeric purity was found to be approximately 91.8% (S)-ent at this stage. The crude material was used directly in the next stage.

### b) S-Benzyl-2-methyl-L(S)-cysteine hydrochloride

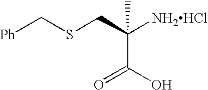

THF (20 mL) and 6M hydrochloric acid (20 mL) were added to the crude product from step (a). The mixture was stirred at RT for 1 h. The organic solvent was evaporated off and the remaining aqueous layer was washed with ether (3×20 mL). The combined organic layers were back extracted with 6M hydrochloric acid (20 mL). The combined aqueous phase was heated at reflux for 16 h. The solvent was evaporated under reduced pressure to yield the title compound as a cream solid (1.10 g, 100%). The enantiomeric purity was shown to be 90.12% (S)-ent (LC; same method as Example 14d).

¹H-NMR (400 MHz, dmso-d6) δ 1.48 (3H, s), 2.87 (1H, d), 2.98 (1H, d), 3.82 (2H, m), 7.3-7.39 (5H, m), 8.48 (3H, s).

### Example 16

### a) Iso-Propyl alaninate hydrochloride

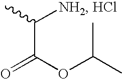

Thionyl chloride (26.70 g, 2241 mmol) was added dropwise to a solution of DL-alanine (10 g, 112 mmol) in iso-propanol (400 mL) at −20° C. The mixture was allowed to come to RT and then heated at reflux for 4 h. The solvent was evaporated to yield the title compound (18.8 g, 100%).

¹H-NMR (300 MHz) δ 1.20-1.22 (6H+3H, each d), 3.46-3.50 (1H, q), 4.01-4.06 (1H, m).

### b) Iso-Propyl N-benzylidenealaninate

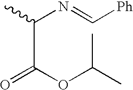

Magnesium sulphate (13.50 g, 112 mmol) and benzaldehyde (4.76 g, 44.8 mmol) were added to a slurry of iso-propyl alaninate hydrochloride (9.40 g, 56 mmol) in DCM (200 mL). Triethylamine (5.6 g, 56 mmol) was added to this mixture which was then stirred for 16 h under a nitrogen atmosphere. More magnesium sulphate (3.30 g, 28 mmol) and triethylamine (0.56 g, 5.6 mmol) were added. The mixture was stirred for 24 h. The mixture was filtered and the filtrate was washed with water (2×50 mL). The organic layer was dried (MgSO₄) and the solvent was evaporated to give the product as a colourless oil (9 g, 76%).

¹H-NMR (400 MHz, CDCl₃,) δ 1.23-1.26 (6H, m), 1.51 (3H, d), 4.08-4.13 (1H, q), 5.03-5.09 (1H, m), 7.36-7.45 (3H, m), 7.76-7.79 (2H, m), 8.31 (1H, s).

### c) S-Benzyl-2-methyl-D(R)-cysteine hydrochloride

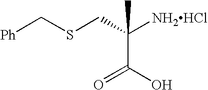

Powdered caesium hydroxide (3.82 g, 22.8 mmol) and chiral phase transfer catalyst (−)-O-allyl-N-(9-anthracenylmethyl)cinchonidinium bromide (0.27 g, 0.45 mmol) were added to iso-propyl N-benzylidenealaninate (1 g, 4.5 mmol) in toluene (10 mL) at −10° C. The mixture was stirred at this temperature for 1 h. The reaction mixture was cooled to −30° C. and a solution of bromomethylsulfanylmethyl-benzene (2.47 g, 11.4 mmol) in toluene (10 mL) was added dropwise. The reaction was stirred at this temperature for 1 h and then allowed to come to RT. The reaction mixture was filtered through celite and washed with toluene (10 mL). The solvent was evaporated to give crude product as a gum. This material was taken up in THF (20 mL) and 6M hydrochloric acid (20 mL) was added. The mixture was stirred at RT for 16 h and then heated at reflux for 4 h. The mixture was cooled to RT and washed with ethyl acetate (20 mL). The aqueous layer was evaporated to dryness under reduced pressure. The residue was taken in toluene (2×20 mL) and then evaporated to dryness under reduced pressure to yield the title compound as a cream solid (1.20 g, 100%). Chiral HPLC analysis showed the product to have 91.72% enantiomeric purity.

¹H-NMR (400 MHz, dmso-d6) δ 1.44 (3H, s), 2.87 (1H, d), 2.98 (1H, d), 3.8-3.85 (2H, m), 7.3-7.39 (5H, m), 8.50 (3H, s).

MS m/z 226 [MH]⁺.

### Example 17

### a) tert-Butyl N-(naphthalen-2-yl-methylidene)alaninate

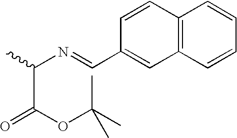

Magnesium sulphate (2.65 g, 22 mmol), 2-naphthaldehyde (1.63 g, 20 mmol) and triethylamine (1.1 g, 10 mmol) were added to a slurry of t-butyl alaninate hydrochloride (DL) (2 g, 11 mmol) in DCM (40 mL). The mixture was stirred for 24 h under a nitrogen atmosphere. Magnesium sulphate (1.32 g, 11 mmol) was added and the reaction was stirred for another 16 h. After analysis, more magnesium sulphate (2.65 g, 22 mmol) was added and mixture was stirred for 3 days at RT. The mixture was filtered and the filtrate was washed with water (2×100 mL). The organic layer was dried with magnesium sulphate, filtered and the solvent was evaporated to give the title compound as a white solid (2.43 g, 78%).

¹H-NMR (300 MHz, CDCl₃) δ 1.46 (9H, s), 1.56 (3H, s), 4.07-4.14 (1H, q), 7.48-7.55 (2H, m), 7.84-7.91 (3H, m), 8.02-8.09 (2H, m), 8.45 (1H, s).

### b) S-Benzyl-2-methyl-D(R)-cysteine hydrochloride

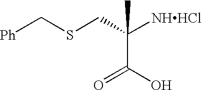

Powdered rubidium hydroxide monohydrate (1.79 g, 17.5 mmol) and chiral phase transfer catalyst (−)-O-allyl-N-(9-anthracenylmethyl)cinchonidinium bromide (0.21 g, 0.35 mmol) were added to tert-butyl N-(naphthalen-2-yl-methylidene)alaninate (1 g, 3.5 mmol) in toluene (10 mL) at −30° C. The mixture was stirred at this temperature for 30 mins. A solution of bromomethylsulfanylmethyl-benzene (1.91 g, 8.8 mmol) in toluene (10 mL) was added dropwise to this mixture. The reaction was stirred at this temperature for 1 h and then stirred at RT for 3 days. The mixture was filtered through celite and washed with toluene (50 mL). The solvent was evaporated to give the protected methyl cysteine as a gum. This material was used directly without isolation or characterisation. The crude product was taken up in a mixture of THF (20 mL) and 6M hydrochloric acid (20 mL). The mixture was stirred at RT for 16 h. The organic solvent was evaporated off and the residue was washed with ethyl acetate (20 mL). The ethyl acetate layer was back extracted with 6M hydrochloric acid (20 mL). The combined aqueous phases were then heated at 70° C. for 10 h. The aqueous layer was washed with ethyl acetate (50 mL) before being evaporated to dryness under reduced pressure. The residue was azeo-dried using toluene (50 mL) to yield the title compound as a cream solid (0.90 g, 83%). Chiral HPLC analysis showed the product to have 94.3% enantiomeric purity.

¹H-NMR (300 MHz, dmso-d6) δ 1.50 (3H, s), 2.90 (1H, d), 3.20 (1H, d), 3.82 (2H, s), 7.3-7.39 (5H, m), 8.52 (3H, s).

MS (ES), m/z 226 [MH]⁺.

1. (5S)-5-[4-(5-Chloro-pyridin-2-yloxy)-piperidine-1-sulfonylmethyl]-5-methyl-imidazolidine-2,4-dione Form G, characterized by having an X-ray powder diffraction pattern comprising specific peaks at 10.1, 16.2, 16.8 and 19.0 °2θ and wherein said XPRD pattern is measured using CuK_{α} radiation.

2. (5S)-5-[4-(5-Chloro-pyridin-2-yloxy)-piperidine-1-sulfonylmethyl]-5-methyl-imidazolidine-2,4-dione Form G, characterized by having an X-ray powder diffraction pattern comprising specific peaks at 9.7, 10.1, 11.5, 12.8, 14.1, 16.2, 16.8 and 19.0 °2θ and wherein said XPRD pattern is measured using CuK_{α} radiation.

3. (5S)-5-[4-(5-Chloro-pyridin-2-yloxy)-piperidine-1-sulfonylmethyl]-5-methyl-imidazolidine-2,4-dione Form G, characterized by having an X-ray powder diffraction pattern substantially the same as that shown in FIG. 1 and wherein said XPRD pattern is measured using CuK_{α} radiation.

4. The compound according to claim 1 that is substantially pure (5S)-5-[4-(5-chloro-pyridin-2-yloxy)-piperidine-1-sulfonylmethyl]-5-methyl-imidazolidine-2,4-dione Form G.

5. The compound according to claim 4 that is at least 99% pure.

6. The compound according to claim 4 that is at least 95% pure.

7. (canceled)

8. A method for in the treatment or prophylaxis of a disease or condition in which inhibition of MMP activity is beneficial, comprising administering to a patient in need thereof a therapeutically effective amount of (5S)-5-[4-(5-Chloro-pyridin-2-yloxy)-piperidine-1-sulfonylmethyl]-5-methyl-imidazolidine-2,4-dione Form G as claimed in claim 1.

9. The method according to claim 8 wherein the disease or condition is an inflammatory disease or condition.

10. The method according to claim 9 wherein the disease is COPD.

11. A pharmaceutical composition comprising (5S)-5-[4-(5-chloro-pyridin-2-yloxy)-piperidine-1-sulfonylmethyl]-5-methyl-imidazolidine-2,4-dione Form G according to claim 1 in admixture with a pharmaceutically acceptable diluent or carrier.

12. A method of treating a disease or condition mediated by metalloproteinase activity, comprising administering to a patient in need thereof a therapeutically effective amount of a pharmaceutical composition of claim 11.

13. A process for the preparation of compound (I) Form G according to claim 1 that comprises crystallisation of (5S)-5-[4-(5-chloro-pyridin-2-yloxy)-piperidine-1-sulfonylmethyl]-5-methyl-imidazolidine-2,4-dione from aqueous alcohol or from aqueous industrial methylated spirits.

14. A process according to claim 13 wherein the (5S)-5-[4-(5-chloro-pyridin-2-yloxy)-piperidine-1-sulfonylmethyl]-5-methyl-imidazolidine-2,4-dione is prepared by reaction of 5-chloro-2-(piperidin-4-yloxy)-pyridine (VI) with ((S)-4-methyl-2,5-dioxo-imidazolidin-4-yl)-methanesulfonyl chloride (V) and wherein compound (VI) is prepared by liberation of the free base from a corresponding salt.

15. A process for the preparation of (S)-5-methyl-5-{[(phenylmethyl)thio]methyl}-imidazolidine-2,4-dione (IV), useful as an intermediate in the synthesis of (5S)-5-[4-(5-chloro-pyridin-2-yloxy)-piperidine-1-sulfonylmethyl]-5-methyl-imidazolidine-2,4-dione, which comprises use of a hydantoinase enzyme to effect ring closure of (RS)-3-benzylsulfanyl-2-methyl-2-ureido-propionic acid (XI).

16. A process according to claim 15 wherein the hydantoinase enzyme is Roche Hydantoinase 1 or Hydantoinase 2.

17. A process for the preparation of (2S)-2-amino-3-benzylthio-2-methylpropionamide (R)-(−)-mandelate hemihydrate, useful as an intermediate in the synthesis of (5S)-5-[4-(5-chloro-pyridin-2-yloxy)-piperidine-1-sulfonylmethyl]-5-methyl-imidazolidine-2,4-dione, which comprises crystallising racemic 2-amino-3-benzylthio-2-methylpropionamide and (R)-(−)-mandelic acid in the presence of water.

18. A process for the preparation of (S)-5-methyl-5-{[(phenylmethyl)thio]methyl}imidazolidine-2,4-dione, useful as an intermediate in the synthesis of (5S)-5-[4-(5-chloro-pyridin-2-yloxy)-piperidine-1-sulfonylmethyl]-5-methyl-imidazolidine-2,4-dione, which comprises enzymatic desymmetrisation of a meso-amide (XIV) using a suitable amidase enzyme.

19. The process according to claim 18 wherein the amidase is Rhodococcus erthoplis amidase.
